(19) United States
(12) Patent Application Publication
(10) Pub. No.: US 2025/0186470 A1
(43) Pub. Date: Jun. 12, 2025

(54) Compositions and Methods Using Lactulose for Treating Disease

(71) Applicant: THE UNIVERSITY OF CHICAGO, Chicago, IL (US)

(72) Inventors: Matthew A. Odenwald, Chicago, IL (US); ERIC G. PAMER, Chicago, IL (US)

(73) Assignee: THE UNIVERSITY OF CHICAGO, Chicago, IL (US)

(21) Appl. No.: 18/896,452

(22) Filed: Sep. 25, 2024

Related U.S. Application Data
(60) Provisional application No. 63/585,175, filed on Sep. 25, 2023.

(51) Int. Cl. A61K 31/7016; A61K 35/745; A61P 31/04; G01N 33/569

(52) U.S. Cl. CPC A61K 31/7016; A61K 35/745; A61P 31/04; G01N 33/56911; G01N 2333/195; G01N 2800/52

(57) ABSTRACT

In some aspects, provided herein are methods for treating a liver disease or associated condition in a subject, and related compositions. In some embodiments, the composition comprises lactulose and a commensal organism, such as a commensal bacterial species. In some embodiments, the patient has been determined to have a microbiome profile and metabolic profile in a fecal sample from the patient, which may be indicative that the patient will benefit from the treatment.

This application claims priority of U.S. Provisional Application No. 63/585,175, filed Sep. 25, 2023, which is hereby incorporated by reference in its entirety.

### BACKGROUND OF THE DISCLOSURE

### I. Field of the Disclosure

This invention relates to the field of microbiology, metabolomics, and medicine.

### II. Background

The incidence of chronic liver disease (CLD) is rising due to increased rates of metabolic syndrome and alcohol use¹⁻⁴. Untreated, all etiologies of CLD converge on the final common endpoint of cirrhosis with similar complications. CLD is typically clinically silent until liver decompensation leads to development of ascites, variceal bleeding or hepatic encephalopathy (HE). Decompensating events are increasingly frequent as CLD progresses, resulting in death or the need for liver transplant^{5,6}. Treatment of advanced CLD is largely supportive and without the ability to significantly modify the overall clinical course. Gut microbial metabolism, including bacterial production of ammonia, exacerbates HE in patients with decompensated cirrhosis, often leading to repeated antibiotic treatment. A current first line treatment for HE is the non-absorbable disaccharide lactulose⁷. The mechanism by which lactulose reduces serum ammonia levels is incompletely defined, and its potential role as a prebiotic that modifies microbiome compositions and metabolic activities remains controversial⁸⁻¹³. Moreover, there are varying reports regarding lactulose's impact on development of complications of CLD, including systemic infections^{14,15}.

The gut microbiome impacts human health and a wide range of diseases. Given the bidirectional communication between the liver and the gut via the portal vein and biliary tree, it is postulated that the microbiome also plays a role in liver disease pathogenesis. Preclinical studies implicate the microbiome as a potential driver of non-alcoholic fatty liver disease (NAFLD) and alcoholic liver disease¹⁶⁻¹⁸. While robust clinical evidence linking the microbiome to progression of liver disease is lacking, multiple observational studies have reported gut microbiome “signatures” of advanced fibrosis and cirrhosis^{9,19-22}. Observational studies have also associated gut microbiome compositions with complications of end stage liver disease^{9,17,23,24}. Different taxa are implicated in these studies, but consistent with studies in non-hepatic diseases, patients with higher burdens of potentially pathogenic taxa (e.g. Proteobacteria or Enterococcus) and lower prevalence of obligate anaerobic commensals (e.g. Lachnospiraceae and Oscillospiraceae) generally have poor prognoses²⁵⁻²⁷.

The consequences of microbiome compositional differences on production of microbiome-derived metabolites remain incompletely defined. Microbe-derived metabolites contribute to intestinal epithelial cell differentiation and barrier formation²⁸⁻³⁰ and regulate mucosal innate and adaptive immune defenses^{27,31,32}. In the case of liver disease, fecal bile acid (BA) profiles have been correlated with progression of NAFLD to non-alcoholic steatohepatitis (NASH) and subsequently advanced fibrosis³³⁻³⁵. Additionally, both total serum BA and specific immunomodulatory circulating BA have been implicated in the progression and prognosis of liver disease³⁶⁻³⁸. While recent studies have identified bacterial species that generate health promoting metabolites, whether these reduce or enhance progression of CLD remains largely unexplored.

Individuals with liver disease can exhibit increased incidence of bacterial infections. The current standard of care for reducing the incidence of infections in patients with liver disease includes lifelong prophylactic antibiotics and/or administration of broad spectrum antibiotics. Such interventions that rely heavily on antibiotics can, over time, increase the incidence of infection with drug-resistant organisms and negatively impact the gut microbiome. The present disclosure addresses these and other needs.

### SUMMARY

In some aspects, the Applicants made certain discoveries that provide advantages for the treatment of liver disease and associated conditions. Certain aspects relate to the findings that patients hospitalized for liver disease have reduced microbiome diversity and a paucity of bioactive metabolites, including short chain fatty acids and bile acid derivatives, that impact immune defenses and epithelial barrier integrity. In some aspects, patients treated with an orally administered but non-absorbable disaccharide lactulose had increased densities of intestinal Bifidobacteria and reduced incidence of systemic infections and mortality.

In some aspects, provided herein is a method of treating a subject, such as a patient. In some aspects, provided herein is a method of treating a drug-resistant pathogen in a patient. In some aspects, provided herein is a method of treating a liver disease in a patient. In some aspects, the method comprises administering a composition comprising lactulose and a commensal organism. In some aspects, the patient is determined to have a specific microbiome profile and a specific metabolic profile in a fecal sample from the patient.

Disclosed are methods of treating a drug-resistant pathogen in a patient, the method comprising administering a composition comprising lactulose and a commensal organism, wherein after the patient has been assayed for both a microbiome profile and a metabolic profile in a fecal sample from the patient.

Also disclosed are methods of treating a liver disease. Also disclosed are methods of measuring a microbiome profile and a metabolite profile in a sample, the method comprising measuring one or more of an Enterococcus sp., a Bifidobacteria sp., a Bacteroidetes sp., a Lachnospiraceae sp., a Proteobacteria sp., and/or a Lactobacillus sp. and measuring 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1.

In various aspects, a fecal sample from a patient has been assayed for one or more specific microbes, including any of the microbes disclosed herein. In various aspects, a fecal sample from a patient has been assayed for one or more metabolites, including any of the metabolites disclosed in Table 1. Assaying such microbes and/or metabolites may determine a microbiome and/or metabolic profile in a patient. Such profiles may be used to determine the patient's responsiveness to a therapy, such as a lactulose and commensal organism therapy.

In certain aspects, the method comprise one or more steps including any of the following: administering a composition comprising lactulose and a commensal organism to a patient; administering lactulose to a patient; administering a commensal organism to a patient; measuring one or more bacteria (including any of the bacteria disclosed herein) in a sample obtained from a patient; measuring one or more metabolites (including any of the metabolites disclosed herein) in a sample obtained from a patient; measuring a metabolic profile in a patient; and measuring a microbiome profile. In certain aspects, the administering is done after the patient has been assayed for both a microbiome profile and a metabolic profile in a fecal sample from the patient.

In certain aspects, the patient has, is suspected of having, has symptoms of, or has been diagnosed with a liver disease. In certain aspects, the drug-resistant pathogen comprises a vancomycin-resistant pathogen. In certain aspects, the drug-resistant pathogen comprises an Enterococcus sp. In certain aspects, the drug-resistant pathogen comprises an Enterococcus faecium. In certain aspects, the commensal organism comprises a Bifidobacteria sp. In certain aspects, the microbiome profile comprises a measured level of an Enterococcus sp., a Bifidobacteria sp., a Bacteroidetes sp., a Lachnospiraceae sp., a Proteobacteria sp., and/or a Lactobacillus sp. In certain aspects, the measured level of one or more of the Enterococcus sp., the Bifidobacteria sp., the Bacteroidetes sp., the Lachnospiraceae sp., the Proteobacteria sp., and/or the Lactobacillus sp. is undetected, zero, or below a detection limit. In certain aspects, the measured level of one or more of the Enterococcus sp., the Bifidobacteria sp., the Bacteroidetes sp., the Lachnospiraceae sp., the Proteobacteria sp., and/or the Lactobacillus sp. is non-zero or above a detection limit. In certain aspects, the metabolic profile comprises a measured level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1. In certain aspects, the metabolic profile comprises a measured level of one or more short chain fatty acids and/or one or more bile acids. In certain aspects, the metabolic profile comprises a measured level of acetate, butyrate, propionate, cholic acid, glycocholic acid, and/or taurocholic acid. In certain aspects, the microbiome profile and the metabolic profile are determined based on a reference profile. In certain aspects, the reference profile is a profile from a healthy individual. In certain aspects, the reference profile is a profile from an individual that was responsive to an administration of lactulose and the commensal organism. In certain aspects, the method comprises measuring a level of an Enterococcus sp., a Bifidobacteria sp., a Bacteroidetes sp., a Lachnospiraceae sp., a Proteobacteria sp., and/or a Lactobacillus sp. in a fecal sample from the patient. In certain aspects, the method comprises measuring a level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1 in a fecal sample from the patient. In certain aspects, the method comprises measuring a level of one or more short chain fatty acids and/or one or more bile acids in a fecal sample from the patient. In certain aspects, the method comprises measuring a level of acetate, butyrate, propionate, cholic acid, glycocholic acid, and/or taurocholic acid in a fecal sample from the patient.

In certain aspects, the lactulose is administered at a dose of between approximately 1-1,000 mg/L or 1-100 g/L. In certain aspects, the commensal organism is administered at a dose of between approximately 1×10⁴ to 9×10⁹ colony forming units of the commensal organism. In certain aspects, the lactulose is administered before, during, and/or after the commensal organism is administered.

In certain aspects, the patient is a human patient.

Also disclosed are compositions comprising lactulose at a concentration of between approximately 1-1,000 mg/L or 1-100 g/L and a commensal organism of approximately 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶ colony forming units. In certain aspects, the commensal organism comprises a Bifidobacteria sp. In certain aspects, the composition is formulated for oral administration. In certain aspects, the composition is formulated for administration to a human patient.

In certain aspects, the method comprises measuring one or more of an Enterococcus sp., a Bifidobacteria sp., a Bacteroidetes sp., a Lachnospiraceae sp., a Proteobacteria sp., and/or a Lactobacillus sp. and measuring 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1. In certain aspects, the sample comprises a fecal sample. In certain aspects, the sample is from a patient that has, is suspected of having, has symptoms of, or is diagnosed with having a drug-resistant pathogen. In certain aspects, the sample is from a patient that has, is suspected of having, has symptoms of, or has been diagnosed with a liver disease. In certain aspects, the drug-resistant pathogen comprises a vancomycin-resistant pathogen. In certain aspects, the drug-resistant pathogen comprises an Enterococcus sp. In certain aspects, the drug-resistant pathogen comprises an Enterococcus faecium. In certain aspects, the microbiome profile comprises a measured level of an Enterococcus sp., a Bifidobacteria sp., a Bacteroidetes sp., a Lachnospiraceae sp., a Proteobacteria sp., and/or a Lactobacillus sp. In certain aspects, the measured level of one or more of the Enterococcus sp., the Bifidobacteria sp., the Bacteroidetes sp., the Lachnospiraceae sp., the Proteobacteria sp., and/or the Lactobacillus sp. is undetected, zero, or below a detection limit. In certain aspects, the measured level of one or more of the Enterococcus sp., the Bifidobacteria sp., the Bacteroidetes sp., the Lachnospiraceae sp., the Proteobacteria sp., and/or the Lactobacillus sp. is non-zero or above a detection limit. In certain aspects, the metabolic profile comprises a measured level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1. In certain aspects, the metabolic profile comprises a measured level of one or more short chain fatty acids and/or one or more bile acids. In certain aspects, the metabolic profile comprises a measured level of acetate, butyrate, propionate, cholic acid, glycocholic acid, and/or taurocholic acid. In certain aspects, the microbiome profile and the metabolic profile are determined based on a reference profile. In certain aspects, the microbiome profile and the metabolic profile are compared to a reference profile. In certain aspects, the reference profile is a profile from a healthy individual. In certain aspects, the reference profile is a profile from an individual that was responsive to an administration of lactulose and the commensal organism. In certain aspects, the method comprises measuring a level of an Enterococcus sp., a Bifidobacteria sp., a Bacteroidetes sp., a Lachnospiraceae sp., a Proteobacteria sp., and/or a Lactobacillus sp. in a fecal sample from the patient. In certain aspects, the sample is from a human.

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” “(x and z) or y,” or “x or y or z.” It is specifically contemplated that x, y, or z may be specifically excluded from an aspect or aspect.

The words “comprising” (and any form of comprising, such as “comprise” and “comprises”), “having” (and any form of having, such as “have” and “has”), “including” (and any form of including, such as “includes” and “include”), “characterized by” (and any form of including, such as “characterized as”), or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

It is contemplated that any aspect discussed in this specification can be implemented with respect to any method or composition of the invention, and vice versa. Furthermore, compositions of the invention can be used to achieve methods of the invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the invention may apply to any other embodiment or aspect of the invention. Furthermore, any composition of the invention may be used in any method of the invention, and any method of the invention may be used to produce or to utilize any composition of the invention. Aspects of an embodiment set forth in the Examples are also aspects that may be implemented in the context of aspects discussed elsewhere in a different Example or elsewhere in the application, such as in the Summary of the Invention, Brief Description of the Drawings, Detailed Description of the Invention, and/or Claims.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C: Fecal samples from hospitalized patients with liver disease display a wide range of microbiome and metabolomic profiles. A total of 847 fecal samples from hospitalized patients with liver disease and 22 healthy donor fecal samples were analyzed by shotgun metagenomics and targeted metabolomics. (A) Relative taxa abundance by shotgun metagenomics is shown for each sample. Metagenomic alpha-diversity was quantified using the inverse Simpson metric, and samples are arranged from left to right in order of increasing metagenomic alpha-diversity. Samples from patients with liver disease were categorized as either low, medium, or high alpha-diversity based on tertiles of inverse Simpson levels. Quantitative targeted metabolite concentrations for each corresponding sample are shown below the metagenomic data. (B) Relative abundance of indicated taxa and (C) select metabolite concentrations were plotted for each tertile of alpha-diversity. Units for SCFA are mM, and units for BA derivatives are in μg/mL. For panels B and C, each point represents a single value where n=283 (low diversity), 282 (medium diversity), 282 (high diversity), and 22 (healthy donor). Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. Individual groups were compared to the healthy donor group. using the Benjamini-Hochberg procedure and represented as follows: *, p<0.05; **, p<0.01; ***, p<0.001; ****, p<0.0001*. There is a wide variation in metagenomic compositions with some samples showing high biodiversity with anaerobic commensal bacteria compared to others that were dominated by a single taxon. Specimens with high alpha-diversity had increasing levels of SCFAs and BA profiles consistent with an ability to deconjugate conjugated primary BA and convert to secondary BA.

FIGS. 2A-2B: Lactulose use is associated with increased Bifidobacteria species abundance and reduced VRE abundance in the absence of systemic antibiotic use. (A) Relative abundance by shotgun taxonomy of one sample per subject is shown (n=22 healthy donors, 262 patients with liver disease). The sample with highest Bifidobacteria abundance for each subject was analyzed. Samples were arranged first by whether they were obtained without lactulose (left) or within 7 days after lactulose administration (right) and then by decreasing Bifidobacterium relative abundance. Among the Bifidobacteriaceae family, a small number of non-Bifidobacteria members are part of the Actinobacteria phylum and are also shown in shades of purple. The antibiotic resistance (vanA) gene was queried and colored based on expression level normalized to gene length (RPKM). Cumulative oral lactulose dose (grams) for 7 days prior to sample collection is shown. Rifaximin, broad-spectrum antibiotics, proton pump inhibitors, and alternative laxatives exposure prior to sample collection is shown. Red=treatment was administered within 7 days prior to the sample being collected; green=treatment not given in this period. The chronicity of liver disease and whether a patient had clinically significant portal hypertension (green=no, red=yes) at time of consent is shown. Stool consistency was categorized as either liquid (yellow), semi-formed (turquoise), or formed (royal blue) for each sample. (B) Relative abundances of Bifidobacteria, Enterococcus, and Proteobacteria were quantified for samples from patients that were not exposed to antibiotics (including rifaximin) in the 7 preceding days. Samples were stratified based on lactulose exposure. One sample per patient was analyzed, and each dot represents a single value (n=47 (lactulose exposure), n=70 (no lactulose exposure). Median and interquartile range are indicated by the horizontal line and box, respectively. The lower vertical line depicts Q1-1.5*IQR and the upper vertical line depicts Q3+1.5*IQR. Statistical comparisons between groups were analyzed using a two-tailed Wilcoxon rank sum test. P-values are adjusted for multiple comparisons using the Benjamini-Hochberg procedure and represented as: *, p<0.05; **, p<0.01; ***, p<0.001; ****, p<0.0001.

FIGS. 3A-3C: Lactulose-mediated Bifidobacteria expansion is associated with significant changes in bioactive fecal metabolites. (A) Volcano plot (log₂fold change vs. log₁₀p-value) of qualitative metabolites comparing samples with low (<10%) vs. high (≥10%) Bifidobacteria abundance after lactulose exposure. P-values were calculated using a two-tailed Wilcoxon rank sum test and are corrected for multiple comparisons using the Benjamini-Hochberg procedure. Values with log 2 fold-change >1 (corresponding to a 2-fold change with a p-value <0.05) were considered significant. (B) select SCFA and BA were quantified. Units for SCFA are mM, and units for BA derivatives are in μg/mL. (C) BA conversion from conjugated-primary BA to primary BA and then to secondary BAs was tested for each sample. Each point represents a molar ratio for an individual sample. For all comparisons (A-C), there is one sample per patient that was chosen based on the highest relative abundance of Bifidobacteria, and sample size was n=87 (lactulose exposure <10% Bifidobacteria) and n=72 (lactulose exposure with ≥10% Bifidobacteria). For panels B and C, each point represents a single sample. Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. P-values are adjusted for multiple comparisons using the Benjamini-Hochberg procedure and represented as follows: *, p<0.05; **, p<0.01; ***, p<0.001; ****, p<0.0001. For both panels B and C, Samples were grouped by whether they had expanded Bifidobacteria in the presence of lactulose. Median and interquartile range are indicated by the line and box, respectively. The lower vertical line depicts Q1-1.5*IQR and the upper vertical line depicts Q3+1.5*IQR. CA: cholic acid; GCA: glycocholic acid; DCA: deoxycholic acid.

FIGS. 4A-4D: Lactulose-mediated Bifidobacteria expansion is associated with exclusion of antibiotic-resistant Enterococcus species. (A) Relative abundance of potentially pathogenic taxa Enterococcus and Proteobacteria were plotted based on lactulose exposure (no lactulose, left; lactulose, right) and relative abundance of Bifidobacteria (<10%, blue; ≥10%, red). Each point represents a single sample with the following sample sizes: total n=262; no lactulose, <10%, n=79; no lactulose, ≥10%, n=24; lactulose, <10%, n=87; lactulose, ≥10%, n=72. Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. P-values are adjusted for multiple comparisons using the Benjamini-Hochberg procedure and represented as follows: *, p<0.05; **, p<0.01; ***, p<0.001; ****, p<0.0001. (B) A human-derived B. longum species was grown in BHIS media (blue) or BHIS supplemented with 50 mM lactulose (red) or 50 mM sucrose (green), and growth curves (OD₆₀₀ over time) are shown. The growth curve shows mean +/− standard deviation for an experiment done in triplicate. This graph is representative of three independent experiments, also done in triplicate, all with consistent results. (C) Schematic of experimental design for B. longum (purple bacilli) and VRE (green cocci) co-culture. B. longum and VRE were grown to steady state and diluted to low density (OD_{600=0.05}) prior to inoculation in either BHIS or BHIS supplemented with 50 mM lactulose. Cultures were inoculated with both bacteria simultaneously (top) or B. longum was given either a 24-hour or 48-hour lead time prior to VRE inoculation (bottom). (D) After 24-hours of co-culture, serial dilutions of three replicates were plated for VRE c.f.u. counts. Each replicate is plotted as an individual point, and the median is represented by the horizontal line. (left=inoculum, middle left=Bifido+VRE, BHIS, middle=Bifido only, BHIS, middle right=Bifido+VRE, lactulose, right=VRE only, lactulose) *, p<0.05 two-tailed student's t-test. The plot is from one experiment that is representative of three independent experiments done in triplicate all with consistent results.

FIGS. 5A-5H: Bifidobacteria expansion and associated metabolite production are associated with decreased incidence of systemic infection and prolonged survival. (A) A Linear discriminant analysis effect size (LEfSe) showing the significant (Wilcoxon rank-sum, two-tailed, p≤0.05) effect sizes of taxa between groups. (B) Acetate (mM) and taurocholic acid (μg/mL) were quantified, and (C) conversion from conjugated-primary BA to primary BA and then to secondary BAs was tested for each sample. Each point represents a molar ratio for an individual sample. (A-C) Sample size: n=101 (no SBP), n=21 (SBP)). (D) LEfSe showing the significant (Wilcoxon rank-sum, two-tailed, p≤0.05) effect sizes of taxa between groups. (E) Acetate (mM) and taurocholic acid (μg/mL) were quantified, and (F) conversion from conjugated-primary BA to primary BA and then to secondary BAs was tested for each sample. Each point represents a molar ratio for an individual sample. (D-F) Sample size: n=227 (no bacteremia), n=19 (bacteremia)). (B,C,E, and F) Each point represents a single sample. Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. P-values are adjusted for multiple comparisons using the Benjamini-Hochberg procedure. Survival curves were stratified based on (G) initial stool sample alpha-diversity (top line=high diversity, middle line=medium diversity, bottom line=low diversity) or (H) lactulose administration and Bifidobacteria expansion (top line=Bifido >10%, bottom line=Bifido <10%) of the initial stool sample. The number at risk for each condition at each 10-day interval is shown below the survival curve. Survival analysis was performed using Kaplan-Meier curves, and the p-value was obtained from a log-rank test. CA: cholic acid, TCA: taurocholic acid, DCA: deoxycholic acid.

FIG. 6: Fecal samples from patients with liver disease display a wide range of metabolomic profiles that correlates with metagenomic alpha-diversity. Initial samples from each patient with full metabolomic profiling (n=237) and healthy donors (n=22) were arranged in order of increasing alpha-diversity as measured by inverse Simpson from left-to-right and shown in the top panel. One third of samples were grouped into low, medium, or high alpha-diversity based on inverse Simpson tertiles. Eighty-two metabolites were analyzed qualitatively for each sample, and the relative concentrations are represented in the pseudocolored heat map. For each compound, a Kruskal-Wallis test was run between the low diversity (n=79), medium diversity (n=79), high diversity (n=79), and healthy donor (n=22) groups. Statistics were adjusted for multiple comparisons using the Benjamini-Hochberg procedure and are color-coded in the far-right column.

FIGS. 7A-7R: The Bifidobacteria expanded cluster has a distinct short chain fatty acid and bile acid profile. (A) A taxonomic UMAP (taxUMAP) was generated using 847 samples from 262 patients with liver disease. Each sample is represented by a single point that is colored based on the most abundant taxon as indicated. Samples with no taxa reaching ≥5% relative abundance were not considered to have a most abundant taxa and were labeled as “other.” (B-G) Samples within the taxUMAP were pseudocolored based on the indicated (B-D) SCFA or (E-G) BA concentrations. (H-R) All 847 stool samples were grouped by most abundant taxon as shown in Panel A. Graphs show the indicated (H-J) SCFA or (K-R) BA concentrations. Each individual point represents a single stool sample with the following sample sizes: n=223 (Enterococcus), 142 (Bacteroidetes), 118 (Bifidobacterium), 91 (Lactobacilluseae), 75 (Lachnospiraceae), 57 (Proteobacteria), 44 (Streptococcus), 17 (Erysipelotrichaceae), 13 (Staphylococcus), 8 (Actinobacteria), 8 (Oscillospiraceae), and 51 (Others). Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. Individual groups were compared to the Bifidobacteria dominated group, the unique cluster in this patient cohort. P-values are adjusted for multiple comparisons using the Benjamini-Hochberg procedure and represented as follows: *, p<0.05; **, p<0.01; ***, p<0.001; ****, p<0.0001.

FIGS. 8A-8C: In the absence of lactulose, Bifidobacteria expansion is associated with modest fecal metabolite changes. (A) Volcano plot (log₂fold change vs. log₁₀p-value) of qualitative metabolites comparing samples with low (<10%) vs. high (≥10%) Bifidobacteria abundance without lactulose exposure. P-values were calculated using a two-tailed Wilcoxon rank sum test and are corrected for multiple comparisons using the Benjamini-Hochberg procedure. Values with log 2 fold-change >1 (corresponding to a 2-fold change with a p-value <0.05) were considered significant. (B) select SCFA and BA were quantified. Units for SCFA are mM, and units for BA derivatives are in μg/mL. (C) BA conversion from conjugated-primary BA to primary BA and then to secondary BAs was tested for each sample. Each point represents a molar ratio for an individual sample. For all comparisons (A-C), there is one sample per patient that was chosen based on the highest relative abundance of Bifidobacteria, and sample size was n=79 (no lactulose exposure <10% Bifidobacteria) and n=24 (no lactulose exposure with ≥10% Bifidobacteria). For panels B and C, each point represents a single sample. Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. P-values are adjusted for multiple comparisons using the Benjamini-Hochberg procedure and represented as follows: *, p<0.05; **, p<0.01; ***, p<0.001; ****, p<0.0001. For both panels B and C, Samples were grouped by whether they had expanded Bifidobacteria in the absence of recent lactulose exposure. Median and interquartile range are indicated by the line and box, respectively. The lower vertical line depicts Q1-1.5*IQR and the upper vertical line depicts Q3+1.5*IQR. CA: cholic acid; GCA: glycocholic acid; DCA: deoxycholic acid.

FIGS. 9A-9N: B. longum acidifies pH, produces acetate, and efficiently hydrolyzes conjugated primary bile acids. B. longum was grown in regular media (blue) or media supplemented with 50 mM lactulose (red) or sucrose (green). (A) pH before and after 24 h of growth (n=3 for 0 h; n=6 for 24 h). (B) SCFA concentrations after 24 h of growth. n=6 all conditions. (C and D) Supernatant BA quantified after 24 h of B. longum growth in media containing 10 μg/ml conjugated primary BA ((C) TCA or (D) GCA). n=4 all conditions. (B-D) *, p<0.05, two-tailed t-test corrected for multiple comparisons. Plots are representative of three independent experiments with ≥3 technical replicates all with consistent results. TCA: taurocholic acid, GCA: glycocholic acid, CA: cholic acid, CDCA: chenodeoxycholic acid, DCA: deoxycholic acid, and LCA: lithocholic acid. (E) Schematic of B. longum gavage, lactulose administration, and stool collection in ex-GF mice. (F) Stool lactulose in GF mice receiving drinking containing 0, 10, 20, or 40 g/L lactulose or in SPF mice with water containing either 0 or 20 g/L lactulose. n=1 for each condition. (G) Stool water content of GF mice receiving water with 0 or 20 g/L lactulose. n=3 for each group. *, p<0.05, two-tailed t-test. (H) Fecal quantitative 16S metagenomics from ex-GF mice colonized with B. longum +/− lactulose in drinking water. n=4 for each group except for day 1 in the water-treated group, when one mouse did not produce a stool sample. n=3 for the lactulose-treated group after day 1 when 1 mouse did not produce stool samples. (I-K) SCFA concentrations before and 10-days after Bifidobacteria inoculation. Each dot represents one sample from each mouse. Bar represents the median. *, p<0.05, two tailed t-test comparing water to lactulose-treated mice at a time point. (L) Primary, (M) conjugated primary, and (N) secondary BA were measured at indicated timepoints. Each dot represents one sample from each mouse. Bar represents the median. *, p <0.05, t-test comparing to time 0 for a given lactulose exposure.

FIGS. 10A-10C: Lactulose promotes Bifidobacteria expansion and favorable metabolomic profile in mice colonized with complex bacterial consortia. (A) Schematic indicating timing of consortia gavage, lactulose administration (20 g/L in drinking water), and stool collection in ex-germ-free mice. 16S: metagenomics; Metab.: targeted, quantitative metabolomics. For CON.1, n=3 mice total (pre- and post-lactulose samples taken); CON.2, n=6 mice (3 without lactulose, 3 with lactulose); and CON.3, n=8 mice (4 without lactulose, 4 with lactulose). (B) 16S metagenomics from stool samples without (left facet within each consortia) and with (right facet within each consortia) lactulose exposure is shown as relative abundance. Concentrations of acetate and taurocholic acid are shown in units of mM below each paired sample. (C) Bifidobacteria relative abundance, acetate concentration (mM), and taurocholic acid concentrations (μg/ml) were measured and compared between mice that were and were not exposed to lactulose. Each dot represents a single stool sample from an individual mouse, and the bar represents the median. *, p<0.05 by one-tailed t-test comparing water to lactulose-treated mice for each consortia.

FIGS. 11A-11F: Bifidobacterium longum supernatant inhibits VRE growth in vitro. (A) Schematic of experimental design for VRE growth in B. longum conditioned media (CM). B. longum was grown in BHIS for 24-hours prior to collecting and filtering the supernatant. VRE growing at steady state was then diluted to a low density (OD_{600=0.05}) prior to inoculating in various dilutions of B. longum conditioned media in fresh BHIS. (B) VRE growth curves in 100%, 50%, 12.5%, and 0% CM with the pH of the 4 concentrations of CM in BHIS shown in the right portion of this panel. (C) The pH of neutral BHIS was acidified from 7.2 to 5.8 using HCl, and the pH of B. longum CM was neutralized from 5.8 to 7.2 with NaOH prior to VRE inoculation. Growth curves for VRE in each of these 4 conditions are shown. (D) VRE was inoculated into either neutral (top panels) or acidified (bottom panels) BHIS containing 0 mM (blue), 30 mM (green), 100 mM (orange), or 300 mM (red) of the indicated SCFA. (E) VRE was grown in minimal media containing either no additive (blue) or 50 mM lactulose, and OD₆₀₀ was measured over time. (F) VRE was grown in BHIS with or without 50 mM lactulose, and OD₆₀₀ was measured over time. All growth curves are depicted with mean +/− standard deviation for technical replicates for each group. Plots are representative of three independent experiments done in at least triplicate, each of which was reproduced on each experimental replicate.

FIGS. 12A-12E: Bifidobacteria expansion and associated metabolite production are associated with decreased incidence of spontaneous bacterial peritonitis (SBP). (A) Flow diagram depicting number of samples and number of patients that were filtered out at each step of analysis for SBP. (B) Taxonomic relative abundance by shotgun metagenomics of stool samples paired to ascites samples is plotted in order of decreasing Bifidobacteria abundance from left to right. Underneath each stool sample is the clinical diagnosis associated with an ascites sample (SBP (red) or not SBP (green)). (C) Volcano plot of normalized metabolite concentrations. P-values were calculated using a two-tailed Wilcoxon rank sum test and are corrected for multiple comparisons using the Benjamini-Hochberg procedure. Values with log 2 fold-change >1 (corresponding to a 2-fold change with a p-value <0.05) were considered significant. (D and E) Quantitative levels for butyrate, propionate, lithocholic acid, and alloisolithocholic acid were compared from samples associated and not associated with SBP. Units for SCFA are mM, and units for BA derivatives are in μg/mL. Each point represents a single sample. Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. There were 101 ascites-adjacent samples not associated with SBP and 21 associated with SBP.

FIGS. 13A-13E: Bifidobacteria expansion and associated metabolite production are associated with decreased incidence of bacteremia). (A) Flow diagram depicting number of samples and number of patients that were filtered out at each step of analysis for bacteremia. (B) Taxonomic relative abundance by shotgun metagenomics of stool samples paired to blood cultures is plotted in order of decreasing Bifidobacteria abundance from left to right. Underneath each stool sample is the clinical diagnosis associated with a given blood culture (bacteremia (red) or no bacteremia (green)). (C) Volcano plot of normalized metabolite concentrations. P-values were calculated using a two-tailed Wilcoxon rank sum test and are corrected for multiple comparisons using the Benjamini-Hochberg procedure. Values with log 2 fold-change >1 (corresponding to a 2-fold change with a p-value <0.05) were considered significant. (D and E) Quantitative levels for butyrate, propionate, lithocholic acid, and alloisolithocholic acid were compared from samples associated and not associated with SBP. Units for SCFA are mM, and units for BA derivatives are in μg/mL. Each point represents a single sample. Median and interquartile range are indicated by the horizontal line and box, respectively. The lower vertical line depicts Q1-1.5*IQR and the upper vertical line depicts Q3+1.5*IQR. Statistical comparisons between individual groups were analyzed using a two-tailed Wilcoxon rank sum test. There were 227 blood culture-adjacent samples not associated with SBP and 19 associated with bacteremia.

### DETAILED DESCRIPTION

To better understand the role of the gut microbiome in the development of CLD complications, including development of infections, aspects herein include a single center observational cross-sectional cohort study of hospitalized patients with liver disease. Metagenomic and metabolomic profiles of fecal samples were correlated with liver disease outcomes. Aspects herein demonstrate that the commonly prescribed disaccharide lactulose preferentially expands Bifidobacteria and, in the absence of systemic antibiotic administration, results in a protective fecal metabolome. Bifidobacteria expansion associates with decreased abundance of antibiotic-resistant pathobionts and improved patient outcomes, including reduced incidence of systemic infections and prolonged survival. Aspects herein also provide insights into the mechanism by which lactulose impacts outcomes of CLD patients and provides rationale for optimizing gut microbiome compositions and functions to minimize complications of liver disease.

Progression of chronic liver diseases is precipitated by hepatocyte loss, inflammation and fibrosis. This process results in the loss of critical hepatic functions, increasing morbidity and the risk of infection. Medical interventions that treat complications of hepatic failure, including antibiotic administration for systemic infections and lactulose treatment for hepatic encephalopathy can impact gut microbiome composition and metabolite production. Certain aspects encompass methods and compositions related to the discovery that patients hospitalized for liver disease have reduced microbiome diversity and a paucity of bioactive metabolites, including short chain fatty acids and bile acid derivatives, that impact immune defenses and epithelial barrier integrity. Certain aspects herein relate to patients treated with the orally administered but non-absorbable disaccharide lactulose have increased densities of intestinal Bifidobacteria and reduced incidence of systemic infections and mortality. Bifidobacteria can metabolize lactulose, produce high concentrations of acetate and acidify the gut lumen, which, in combination, can reduce the growth of antibiotic-resistant bacteria such as Vancomycin-resistant Enterococcus faecium. Certain aspects herein show that lactulose and Bifidobacteria serve as a synbiotic to reduce rates of infection in patients with severe liver disease.

### I. Sample Preparation

In some aspects, the methods provided herein include obtaining a sample. In some embodiments, the sample is from a subject, such as a patient. In some embodiments, the sample is a fecal sample (i.e. a sample consisting of or containing feces), such as a stool sample. The fecal sample can be obtained or collected by any suitable means. In some embodiments, the sample is from a subject having or suspected of having liver disease and/or associated complications. In some embodiments, the sample is analyzed to determine whether a specific microbiome profile and/or specific metabolic profile is present in the sample, for example in connection with the methods provided herein. In some embodiments, the sample is prepared for analysis of microbiome composition, such as by nucleic acid sequencing (e.g. DNA sequencing for assessing microbiome composition). In some embodiments, the sample is prepared for analysis of metabolites, such as by mass spectrometry (e.g. gas chromatography-mass spectrometry (GC-MS) and/or liquid chromatography-mass spectrometry (LC-MS)).

The methods of obtaining provided herein may include methods of fecal/stool collection, biopsy such as fine needle aspiration, core needle biopsy, vacuum assisted biopsy, incisional biopsy, excisional biopsy, punch biopsy, shave biopsy or skin biopsy. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, stool, or saliva. In certain aspects of the current methods, any medical professional such as a doctor, nurse or medical technician may obtain a biological sample for testing. Yet further, the biological sample can be obtained without the assistance of a medical professional. For example, the sample can be obtained by the subject, for example using a provided kit, such as for fecal sample collection.

The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

In some cases, the sample may be obtained, stored, or transported using components of a kit of the present methods. In some cases, multiple samples, such as multiple fecal samples may be obtained for analysis by the methods described herein. In some cases, multiple samples (such as two or more fecal samples) may be obtained at the same or different times. Samples may be obtained at different times and may be stored and/or analyzed by different methods. In some embodiments, multiple samples may be obtained by the methods herein to ensure a sufficient amount of biological material.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a fecal sample.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

### II. Compositions

In some aspects, provided herein are compositions, such as therapeutic compositions and pharmaceutical compositions. In some embodiments, the composition comprises lactulose and a commensal organism. The compositions can be used in connection with any of the methods provided herein, for example in methods of treating a drug-resistant pathogen in a patient, and/or methods of treating liver disease in a patient.

In some embodiments, the composition comprises a prebiotic (e.g. the lactulose). In some embodiments, the composition comprises a probiotic (e.g. the commensal organism). In some embodiments, the composition comprising lactulose and a commensal organism is a synbiotic. In some aspects, a synbiotic is a composition comprising a prebiotic (i.e. the lactulose) and a probiotic (i.e. the commensal organism). In some aspects, a prebiotic is a composition, such as a compound, that fosters growth or activity of beneficial microorganisms. In some aspects, lactulose can serve as the prebiotic in the synbiotic. In some aspects, a probiotic is a beneficial microorganisms, such as the commensal bacteria. Thus, in some aspects, the commensal bacteria can serve as the probiotic of the synbiotic.

In some aspects, lactulose is a sugar which may have therapeutic benefits in connection with the compositions and methods as described herein. In some aspects, lactulose can be administered by any suitable means to the subject, including orally and/or rectally. In some embodiments, the lactulose is administered orally. In some embodiments, the lactulose is administered rectally. In some embodiments, the composition comprises any suitable amount and/or concentration of lactulose to achieve the benefits described herein. In some embodiments, the lactulose is administered at a dose of between approximately 1-1000 mg/L or 1-100 g/L. In some embodiments, the lactulose can be administered before, during, and/or after the commensal organism is administered.

In some aspects, the commensal organism is any suitable commensal organism which may have therapeutic benefits in connection with the compositions and methods as described herein. In some embodiments, the commensal organism is bacteria. In some embodiments, the commensal organism is any suitable Bifidobacteria species (sp). For example, in some embodiments, the commensal organism is Bifidobacterium longum. In some aspects, the commensal organism can be administered by any suitable means to the subject, including orally and/or rectally. In some embodiments, the commensal organism is administered orally. In some embodiments, the commensal organism is administered rectally. In some embodiments, the composition comprises any suitable amount and/or concentration of commensal organism to achieve the benefits described herein. In some embodiments, the commensal organism is administered at a dose of between approximately 1×10⁴ to 9×10⁹ colony forming units of the commensal organism. In some embodiments, the commensal organism can be administered before, during, and/or after the lactulose is administered.

The composition can be administered by any suitable means to the subject, including orally and/or rectally. In some embodiments, the composition is administered orally. In some embodiments, the composition is administered rectally. It can be seen that the composition comprises both a lactulose component and a commensal bacteria component. In some embodiments, the two components of the composition may be administered together or separately. For example, in some embodiments, the lactulose and commensal organism can be delivered simultaneously by the same method (e.g. either orally or rectally). In other embodiments, the lactulose and commensal organism can be delivered sequentially and/or by different methods (e.g. the lactulose can be delivered orally and the commensal organism can be delivered rectally).

Certain aspects relate to compositions, such as therapeutic compositions, comprising commensal organisms. The commensal organism can be a Bifidobacteria species (sp). In some aspects, the composition comprises bacteria, such a Bifidobacteria sp., in a unit dosage. The unit dosage may be any dosage sufficient for the desired effect of the therapeutic composition. In some aspects the unit dosage comprises between 1×10³ to 9×10¹⁶ colony forming units (CFU) of the commensal organism. In some aspects the unit dosage comprises between 1×10⁴ to 9×10⁹ colony forming units (CFU) of the commensal organism. In some aspects the unit dosage comprises at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴. 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the commensal organism. In another aspect, the disclosure relates to compositions comprising an isolated or purified population of the commensal organism, such as a Bifidobacteria sp. Therapeutic compositions and methods of administering the commensal organism may involve such unit dosages. Moreover, a unit dosage may be given multiple times over a time period as discussed below.

Certain aspects relate to compositions comprising a prebiotic, such as lactulose. The compositions of the present disclosure may further comprise one or more additional prebiotics known in the art. In some embodiments, the lactulose of the composition may be replaced by a suitable substitute prebiotic.

The compositions can be formulated for administration, including as pharmaceutical formulations, e.g., formulated for oral administration; suppository administration; or injection such as via the intravenous, intramuscular, subcutaneous, or intraperitoneal routes. Such compositions can be prepared as either liquid solutions or suspensions; solid forms suitable for use to prepare solutions or suspensions upon the addition of a liquid prior to injection can also be prepared; and, the preparations can also be emulsified.

In certain aspects, the composition, which may include lactulose and the commensal organism, is formulated for oral administration. The formulation for oral administration may comprise a pill, capsule, suspension, drink, or the like. In some aspects, the composition or a component of the composition (e.g. the lactulose) is administered through food.

In some aspects, the composition or a component thereof is a fecal transplant. In some aspects, the fecal matter is administered in a dose of 50 g. In some embodiments, the fecal matter is administered in a dose of at least, at most, or exactly 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, or 400 g (or any derivable range therein). In certain aspects, the fecal transplant comprises fecal matter collected from a patient that has not received a stem cell therapy or antibiotic in 1, 2, 3, 4, 5, 6, 7, 8, 9, 10 days, weeks, months, and/or years (and any range derivable therein) prior to collecting the fecal matter. In certain aspects, the fecal transplant comprises fecal matter that comprises a measurable amount of the commensal organism (e.g. Bifidobacteria sp.). In certain aspects, the fecal transplant comprises fecal matter that comprises a therapeutically effective amount of the commensal organism.

The pharmaceutical formulations suitable for injectable use include sterile aqueous solutions or dispersions; formulations including, for example, aqueous propylene glycol; and sterile powders for the extemporaneous preparation of sterile injectable solutions or dispersions. In certain aspects, the formulation is stable under the conditions of manufacture and storage and preserved against the contaminating action of non-therapeutic microorganisms.

A pharmaceutical composition or formulation can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of unintended microorganisms can be brought about by various anti-bacterial and anti-fungal agents, for example, parabens, chlorobutanol, phenol, sorbic acid, thimerosal, and the like. In certain aspects, the formulation includes isotonic agents, for example, sugars or sodium chloride. Prolonged absorption of the injectable compositions can be brought about by the use in the compositions of agents delaying absorption, for example, aluminum monostearate and gelatin.

Injectable solutions may be prepared by incorporating the active compounds in the required amount in the appropriate solvent with various other ingredients enumerated above, as required. In certain aspects encompassing powders for the preparation of injectable solutions, the therapeutic composition(s) are vacuum-dried and/or freeze-dried, which yield a powder of the active ingredient, plus any additional desired ingredient.

The present disclosure also provides a pharmaceutical composition comprising one or more microbial cultures of the commensal organism. The commensal organism therefore can be present in the dose form as live bacteria, whether in dried, lyophilized, or sporulated form. This may be preferably adapted for suitable administration; for example, in tablet or powder form, potentially with an enteric coating, for oral treatment.

In particular aspects, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure are preferably formulated for oral administration, other routes of administration can be employed, however, including, but not limited to, intracolonic, subcutaneous, intramuscular, intradermal, transdermal, intraocular, intraperitoneal, mucosal, vaginal, rectal, and intravenous.

In another aspect, the disclosed composition may be prepared as a suppository. The suppository may include but is not limited to the commensal organism and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the composition may be prepared as a tablet. The tablet may include the commensal organism and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the composition may be formed as food or drink or, alternatively, as an additive to food or drink, wherein an appropriate quantity of the commensal organism is added to the food or drink to render the food or drink the carrier.

In some aspects, the composition may further comprise a food or a nutritional supplement effective to stimulate the growth of the commensal organism in the gastrointestinal tract of the subject. In some aspects, the nutritional supplement is produced by a bacterium associated with a healthy human gut microbiome.

### III. Administration and Methods of Treatment

In some aspects, provided herein is a method of treating a subject, such as a patient. In some aspects, provided herein is a method of treating a drug-resistant pathogen in a patient. In some aspects, provided herein is a method of treating a liver disease in a patient. In some aspects, the method comprises administering a composition comprising lactulose and a commensal organism. In some aspects, the patient is determined to have a specific microbiome profile and a specific metabolic profile in a fecal sample from the patient.

Certain aspects concern the administration of therapies and therapeutic compositions, including any of the compositions described herein that include lactulose and a commensal organism. The therapies may be administered in any suitable manner. The therapy provided herein may comprise administration of different components of the composition, such as a first component comprising the commensal organism and a second component comprising the lactulose. The first and second components may be administered sequentially (at different times) or simultaneously (at the same time). In some aspects, the first and second components are administered as separate compositions. In some aspects, the first and second components are administered as the same composition. In some aspects, the first component and the second component are administered substantially simultaneously. In some aspects, the first component and the second component are administered sequentially. In some aspects, the first component is administered before administering the second component. In some aspects, the first component is administered after administering the second component.

In some aspects, the composition may be administered to a subject for a therapeutic purpose. For example, the composition may be administered to treat a drug-resistant pathogen, and/or to treat liver disease or a symptom thereof. In some embodiments, the subject has or is suspected of having liver disease. In some embodiments, the subject has or is suspected of having a pathogen, such as a drug-resistant pathogen. In some embodiments, the subject has or is suspected of having liver disease. In some embodiments, the pathogen is a pathogen with antibiotic resistance. In some embodiments, the pathogen is a vancomycin-resistant pathogen. In some embodiments, the drug-resistant pathogen comprises an Enterococcus sp. In some embodiments, the drug-resistant pathogen comprises an Enterococcus faecium.

In some embodiments, administering the composition provides a therapeutic benefit. For example, in some embodiments, administering the composition may inhibit the growth of pathogens, such as any of the pathogens provided herein, including drug-resistant pathogens. In some embodiments, administration can lead to the production of metabolites (e.g. by the commensal organism of the composition or other commensal organisms present in the microbiome of the subject), which inhibit colonization and growth of pathogens. In some embodiments, administering the composition can reduce the incidence of infections. In some embodiments, administering the composition can prolong patient survival (e.g. survival in patients with liver disease).

In some aspects, system infections in patients with liver disease are common precipitants of multiple decompensating events (e.g. hepatic encephalopathy). Thus, in some embodiments, administration of the composition can be used to prevent and or prophylactically treat certain outcomes, such as hepatic encephalopathy.

In certain aspects, composition is administered in an amount that prevents, reduces the severity of, or treats a disease or disorder. Such amount may be referred to herein as a therapeutically effective amount.

The compositions or components thereof of the disclosure may be administered by the same route of administration or by different routes of administration. In some aspects, the composition is administered intracolonically, intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the patient, the patient's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a single dose of the composition is administered. In some aspects, multiple doses of the composition are administered. In some aspects, the composition is administered at a dose of between 1×10⁴ to 9×10⁹ CFUs of the commensal organism, or any range derivable therein. In some aspects, the first therapeutic composition is administered at a dose of at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10^{13 5}×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria.

In some aspects, a single dose of the composition comprising lactulose is administered. In some aspects, the lactulose is administered at a dose of 1 μg/kg to 1 mg/kg, or any range derivable therein, or between approximately 1-1,000 mg/L or 1-100 g/L, or any range derivable therein.

The quantity to be administered, both according to number of treatments and unit dose, depends on the treatment effect desired. An effective dose is understood to refer to an amount necessary to achieve a particular effect. Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition or components thereof, e.g., 2, 3, 4, 5, 6 or more (and any range derivable therein) administrations. The administrations can be at 1, 2, 3, 4, 5, 6, 7, 8, to 5, 6, 7, 8, 9, 10, 11, 12, 13, 14 day, week, or month intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

Administration of the compositions will typically be via any common route. This includes, but is not limited to oral, suppository, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

The desired dose of the composition of the present disclosure may be presented in multiple (e.g., two, three, four, five, six, or more) sub-doses administered at appropriate intervals throughout the day.

Upon formulation, solutions will be administered in a manner compatible with the dosage formulation and in such amount as is therapeutically or prophylactically effective. The formulations are easily administered in a variety of dosage forms, such as the type of injectable solutions described above.

### IV. Microbiome and Metabolic Profiles

In some aspects, the compositions and methods provided herein are administered to a subject (e.g. patient) having a specific microbiome profile. In some aspects, the compositions and methods provided herein are administered to a subject (e.g. patient) having a specific metabolic profile. In some aspects, the compositions and methods provided herein are administered to a subject (e.g. patient) having a specific microbiome profile and a specific metabolic profile. In some embodiments, the specific microbiome profile and the specific metabolic profile are determined based on a reference profile. In some embodiments, the reference profile is a profile from a healthy individual. In some embodiments, the reference profile is a profile from an individual that was responsive to an administration of lactulose and the commensal organism.

In some embodiments, the subject has a specific microbiome profile. In some embodiments, the specific microbiome profile may be an indicator that the subject may benefit from being administered with the compositions provided herein comprising lactulose and a commensal organism. In some embodiments, the microbiome profile is the amount and/or concentration (i.e. level) of one or more species or categories of commensal organisms (e.g. bacteria) in a fecal sample of the subject. In some embodiments, the specific microbiome profile comprises a measured level of an Enterococcus sp., a Bifidobacteria sp., a Bacteroidetes sp., a Lachnospiraceae sp., a Proteobacteria sp., and/or a Lactobacillus sp. In some embodiments, the measured level of one or more of the Enterococcus sp., the Bifidobacteria sp., the Bacteroidetes sp., the Lachnospiraceae sp., the Proteobacteria sp., and/or the Lactobacillus sp. is undetected, zero, or below a detection limit. In some embodiments, the measured level of one or more of the Enterococcus sp., the Bifidobacteria sp., the Bacteroidetes sp., the Lachnospiraceae sp., the Proteobacteria sp., and/or the Lactobacillus sp. is non-zero or above a detection limit.

In some embodiments, the subject has a specific metabolic profile. In some embodiments, the specific metabolic profile may be an indicator that the subject may benefit from being administered with the compositions provided herein comprising lactulose and a commensal organism. In some embodiments, the metabolic profile comprises a measured level of one or more metabolites. In some embodiments, the specific metabolic profile comprises a measured level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1. In some embodiments, the specific metabolic profile comprises a measured level of one or more short chain fatty acids and/or one or more bile acids. In some embodiments, the specific metabolic profile comprises a measured level of acetate, butyrate, propionate, cholic acid, glycocholic acid, and/or taurocholic acid.

TABLE 1
Exemplary Metabolites for Metabolic Profiling

| Metabolite | Category |
|---|---|
| Valerate | Short-Chain FA |
| Propionate | Short-Chain FA |
| Butyrate | Short-Chain FA |
| Hexanoate | Short-Chain FA |
| Acetate | Short-Chain FA |
| Crotonate | Short-Chain FA |
| 2-Methylbutyrate | Branched-Chain FA |
| Isobutyrate | Branched-Chain FA |
| 4-Methylvalerate | Branched-Chain FA |
| 5-Aminovalerate | Aminated FA |
| 3-Aminoisobutyrate | Aminated FA |
| Palmitate | Long-Chain FA |
| Succinate | Dicarboxylic Acid |
| Itaconate | Dicarboxylic Acid |
| Cysteine | Amino Acid |
| Methionine | Amino Acid |
| Alanine | Amino Acid |
| Tyrosine | Amino Acid |
| Tryptophan | Amino Acid |
| Phenylalanine | Amino Acid |
| Valine | Amino Acid |
| Glycine | Amino Acid |
| Glutamate | Amino Acid |
| Isoleucine | Amino Acid |
| Leucine | Amino Acid |
| Aspartate | Amino Acid |
| Lysine | Amino Acid |
| Proline | Amino Acid |
| Cholic Acid | 1° Bile Acid (BA) |
| Chenodeoxycholic Acid | 1° BA |
| Lithocholic Acid | 2° Bile Acid (BA) |
| 3-Oxolithocholic Acid | 2° BA |
| Deoxycholic Acid | 2° BA |
| Isolithocholic Acid | 2° BA |
| Isodeoxycholic Acid | 2° BA |
| 12-Oxolithocholic Acid | 2° BA |
| Hyodeoxycholic Acid | 2° BA |
| Allolithocholic Acid | 2° BA |
| Alloisolithocholic Acid | 2° BA |
| Alpha-Muricholic Acid | 2° BA |
| 3-epicholic/omega-Muricholic Acid | 2° BA |
| Beta-Muricholic Acid | 2° BA |
| Ursodeoxycholic Acid | 2° BA |
| 12-Oxochenodeoxycholic Acid | 2° BA |
| 7-Oxodeoxycholic Acid | 2° BA |
| 3-Oxocholic Acid | 2° BA |
| Allocholic Acid | 2° BA |
| 3-Deoxycholic Acid | 2° BA |
| Gamma-Muricholic Acid | 2° BA |
| Glycolithocholic Acid | Conjugated Bile Acid (BA) |
| Glycodeoxycholic Acid | Conjugated BA |
| Taurochenodeoxycholic Acid | Conjugated BA |
| Glycohyodeoxycholic Acid | Conjugated BA |
| Taurohyodeoxycholic Acid | Conjugated BA |
| Tauroursodeoxycholic Acid | Conjugated BA |
| Taurocholic Acid | Conjugated BA |
| Glycoursodeoxycholic Acid | Conjugated BA |
| Glycocholic Acid | Conjugated BA |
| Glycochenodeoxycholic Acid | Conjugated BA |
| Glycodehydrocholic Acid | Conjugated BA |
| N-Acetylserotonin | Indole |
| Indole-3-Propionate | Indole |
| Indole-3-Carboxaldehyde | Indole |
| Indole-3-Acetate | Indole |
| Indole | Indole |
| Indole-3-Acrylate | Indole |
| Tryptamine | Indole |
| Indole-3-Lactate | Indole |
| Methylserotonin | Indole |
| Tryptophol | Indole |
| Indole-3-Acetamide | Indole |
| P-Cresol | Phenolic Aromatic |
| Tyramine | Phenolic Aromatic |
| Desaminotyrosine | Phenolic Aromatic |
| Phenol | Phenolic Aromatic |
| Anthranilic Acid | Kynurine Pathway |
| Picolinic Acid | Kynurine Pathway |
| Kynurenine | Kynurine Pathway |
| Quinolinic Acid | Kynurine Pathway |
| Kynurenic Acid | Kynurine Pathway |
| Niacin | Vitamin |
| Biotin | Vitamin |
| Folate | Vitamin |

In some embodiments of the method, the method can comprise analyzing the microbiome of the patient, such as a microbiome profile in the patient. For example, in some embodiments, the method comprises measuring a level of an Enterococcus sp., a Bifidobacteria sp., a Bacteroidetes sp., a Lachnospiraceae sp., a Proteobacteria sp., and/or a Lactobacillus sp. in a fecal sample from the patient.

In some embodiments of the method, the method can comprise analyzing metabolites in the patient, such as a metabolic profile. For example, in some embodiments, the method comprises measuring a level of one or more metabolites in the patient, such as in a fecal sample from the patient. In some embodiments, the method comprises measuring a level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1 in a fecal sample from the patient. In some embodiments, the method comprises measuring a level of one or more short chain fatty acids and/or one or more bile acids in a fecal sample from the patient. In some embodiments, the method comprises measuring a level of acetate, butyrate, propionate, cholic acid, glycocholic acid, and/or taurocholic acid in a fecal sample from the patient.

In some aspects, metabolic profile can be analyzed according to any suitable method. For example, the levels of the measured metabolites can be analyzed by methods such as mass spectrometry (e.g. gas chromatography-mass spectrometry (GC-MS) and/or liquid chromatography-mass spectrometry (LC-MS)).

In some aspects, the methods relate to obtaining a microbiome profile of a patient. In some aspects, obtaining a microbiome profile comprises the steps of or the ordered steps of: i) obtaining a sample obtained from a subject (e.g., a human subject), ii) isolating one or more bacterial species from the sample, iii) isolating one or more nucleic acids from at least one bacterial species, iv) sequencing the isolated nucleic acids, and v) comparing the sequenced nucleic acids to reference nucleic acid sequences. When performing the methods necessitating genotyping, any genotyping assay can be used. For example, this can be done by sequencing the 16S or the 23S ribosomal subunit or by metagenomics shotgun DNA sequencing associated with metatranscriptomics.

In some aspects, obtaining the microbiome profile of a patient is used to monitor the need of administering the therapeutic compositions described herein to the patient. In certain aspects, obtaining the microbiome profile of a patient is used to monitor the efficacy of the therapeutic compositions administered to the patient, including monitoring the concentration of the commensal organism in the profile. In certain aspects, the patient is or is not administered a therapeutic composition based on the obtained microbiome profile of the patient. In certain aspects, the patient is administered a therapeutic composition because the obtained microbiome profile is the specific microbiome profile, for example which can be determined based on a reference profile of a healthy individual. In certain aspects, the patient is administered a therapeutic composition because the obtained microbiome profile has an increased and/or decreased amount of one or more bacteria species and/or genus of bacteria when compared to a standard.

In certain aspects, the standard for comparison of the microbiome profile is a microbiome profile from a healthy individual. The healthy individual may be a patient that does not have liver disease and/or a drug-resistant pathogen infection. In certain aspects, the healthy individual is a patient that does not have a diagnosed intestinal disorder.

Methods for determining microbiome composition may include one or more microbiology methods such as sequencing, next generation sequencing, wester blotting, comparative genomic hybridization, PCR, ELISA, etc.

In some aspects, the patient receiving a therapeutic composition, including any therapeutic composition described herein, has a higher abundance of at least one bacteria species and/or genus of bacteria in comparison to a healthy individual. In certain embodiments, the patient has a higher abundance of at least one bacteria species and/or genus of bacteria, such as Enterococcus. In some embodiments, the drug-resistant pathogen comprises a vancomycin-resistant pathogen. In some embodiments, the drug-resistant pathogen comprises an Enterococcus sp., such as Enterococcus faecium.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Patient Recruitment and Disease Characteristics

The inventors enrolled 356 hospitalized patients with liver disease. Of these 356 patients, 262 (73.6%) produced 847 stool samples that were analyzed by shotgun metagenomics and paired targeted metabolomics. Demographics and admission disease characteristics of patients who produced samples are shown (Table A1). Most patients enrolled were hospitalized with decompensated cirrhosis (n=196, 74.8%), and of those, alcohol use was the most common liver disease etiology (n=124, 63.3%). The cohort had a median model of end stage liver disease MELD-sodium (MELD-Na) score of 18.69, which was higher in patients with decompensated cirrhosis compared to other disease states. Of the 262 patients with samples, 183 (69.8%) had clinically significant portal hypertension, which was more common in patients with decompensated cirrhosis. Over half of enrolled patients had end organ dysfunction on admission, which was defined using NACSELD criteria³⁹. Acute on chronic liver failure (ACLF) (i.e. 2 or more organ failures) was present in 14.7% of patients with decompensated cirrhosis upon enrollment. End organ dysfunction was more prevalent in patients with decompensated cirrhosis, only severe HE was statistically significant. Consistent with the poor prognosis suggested by enrollment disease characteristics, patients with decompensated cirrhosis had a 20.9% 90-day mortality rate.

### Microbiome and Metabolite Profiles of Liver Disease Patients

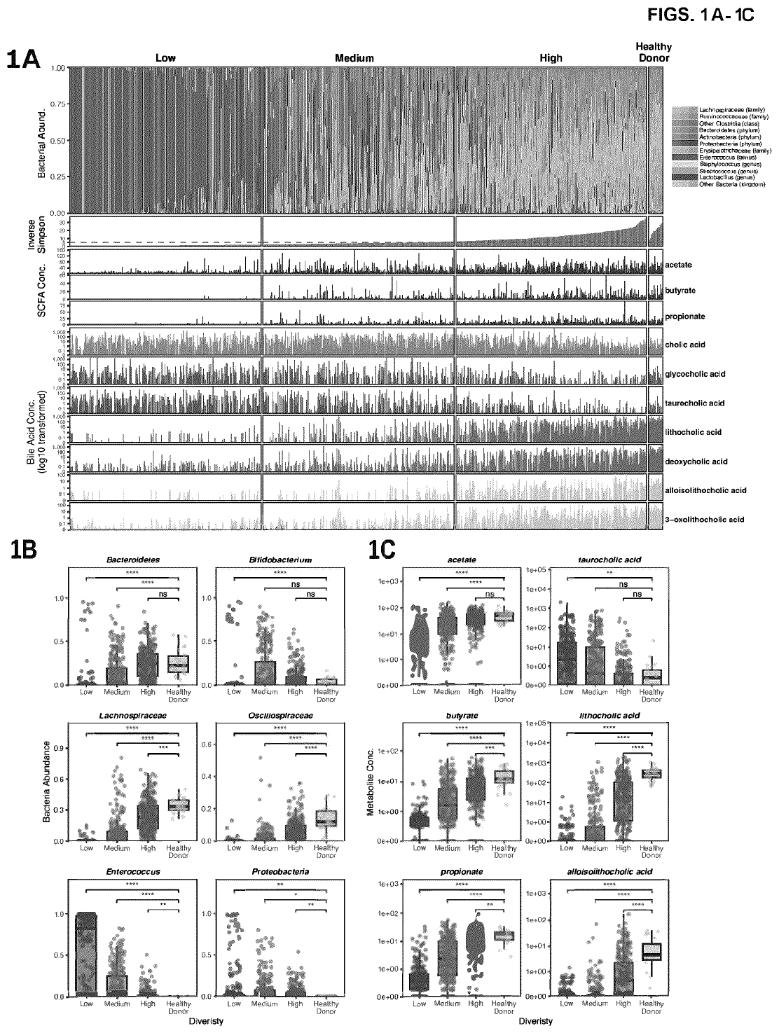

To determine the fecal microbiome compositions in patients with liver disease, the inventors performed shotgun sequencing on DNA from 847 fecal samples collected from 262 liver disease patients and 22 healthy donors (median age 35.00 years-old, IQR 25.75-42.50). The inventors used MetaPhlan4 to assign taxonomic compositions and inverse Simpson to assess microbial diversity (FIG. 1A). Most fecal samples have low taxonomic alpha-diversity (FIG. 1A, inverse Simpson range: 1.00-37.55, mean=6.35, median=3.85) with expansion of bacterial taxa belonging to the Enterococcus genus and Enterobacteriaceae family, common hallmarks of dysbiosis associated with antibiotic exposure^{25,40}. Pathobiont expansion was detected in fecal samples across all alpha-diversity tertiles in patients with liver disease compared to healthy controls (FIG. 1A, B). Marked expansion of the genus Bifidobacteria (FIG. 1A, 1B) was also detected in a subset of samples across alpha-diversity groups. While Bifidobacteria expansion is common in breast fed infants, it is rarely detected in adults, suggesting that this may be liver disease-specific.

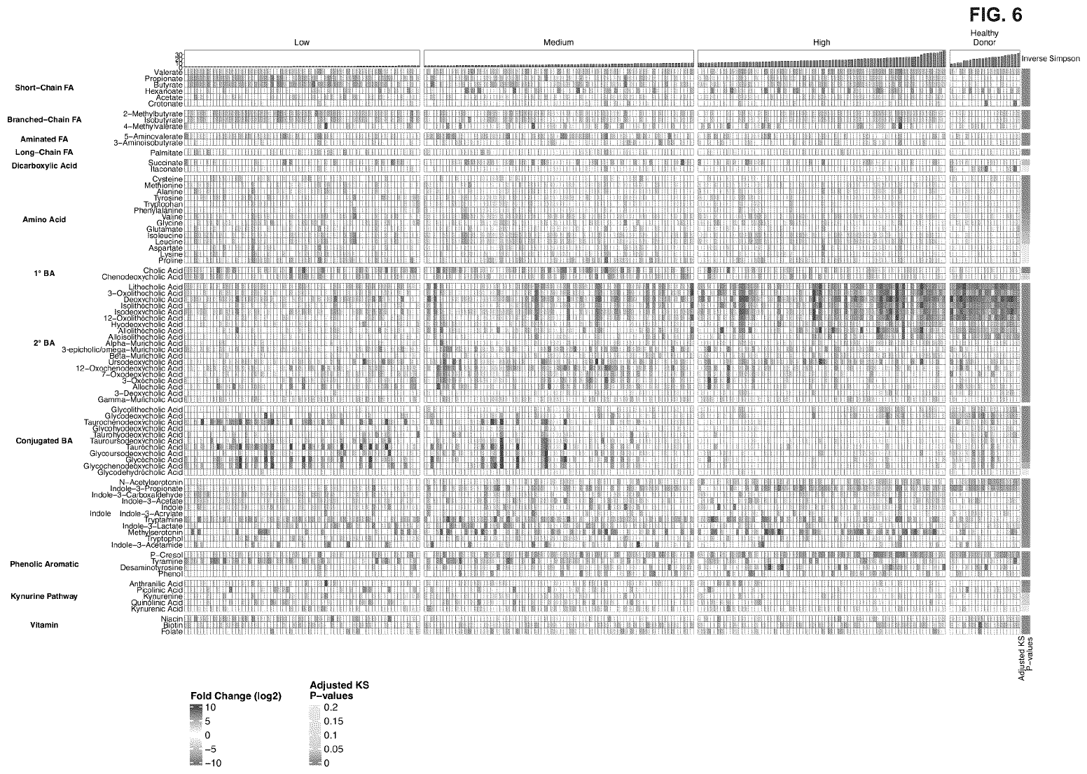

While microbiome-derived or modified metabolites mediate many beneficial impacts on mucosal immune defenses and epithelial barrier functions, little is known about their production in liver disease patients harboring vastly different microbial populations. The inventors used targeted GC- and LC-MS to determine relative amounts 82 metabolites in the dataset (FIG. 6). Ranking fecal samples by alpha-diversity demonstrates a correlation between microbiome diversity and relative amounts of short chain fatty acids (SCFAs), branched chain FAs, aminated FAs, secondary BA, and various indole compounds and tryptophan metabolites. As expected, low diversity fecal samples have markedly reduced secondary BA levels but also reduced concentrations of SCFAs and a subset of indole compounds.

The inventors next quantified concentrations of SCFAs and primary and secondary BA in fecal samples from patients with liver disease. Higher proportions of commensal anaerobes and alpha-diversity coincided with higher concentrations of SCFA, secondary and modified secondary BA and reduced concentrations of conjugated primary BA (FIG. 1A-C). While high diversity fecal samples from patients with liver disease had microbial taxonomic and metabolomic profiles that approached those seen in healthy donors, there was reduced representation of Lachnospiraceae and Oscillospiraceae taxa, SCFA production, and secondary BA synthesis (FIG. 1B, C). Thus, while nearly all patients hospitalized with liver disease have reduced microbiome diversity and microbiome-derived metabolite concentrations, the findings demonstrate a steep gradient extending from relatively minor to absolute loss of microbiome compositions and health-associated metabolites.

Fecal Metabolites Correlate with Distinct Microbial Taxa

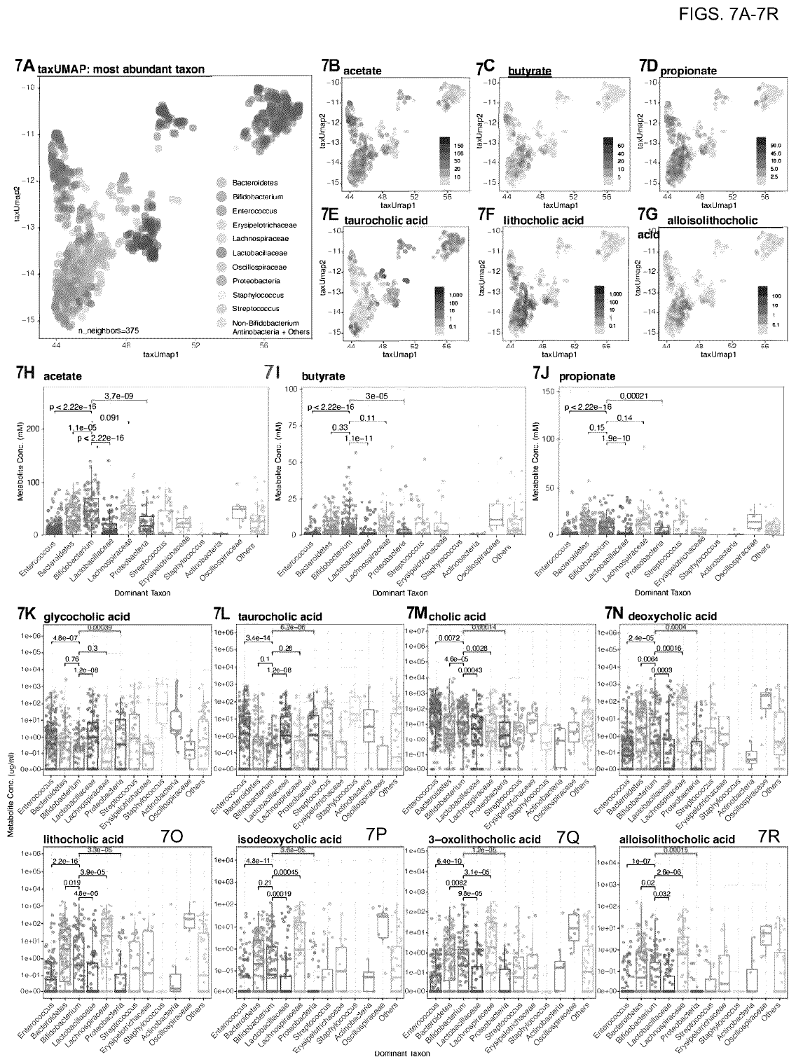

To associate fecal metabolite concentrations with microbiome compositions, fecal samples were plotted on a taxonomic Uniform Manifold Approximation and Projection (taxUMAP) and assigned to one of twelve taxonomic groups based on the most prevalent taxons (FIG. 7A). Samples dominated by Enterococci, Proteobacteria, Actinobacteria (predominantly Bifidobacteria) and Lactobacilli form distinct clusters on the taxUMAP. Overlaying fecal metabolite concentrations demonstrates that Enterococcus, Proteobacteria and Lactobacillus clusters have lower SCFA concentrations compared to clusters dominated by Bifidobacteria, Lachnospiraceae or Bacteroidetes (FIGS. 7A-D, H-J). Bifidobacteria-expanded samples had higher acetate concentrations (FIGS. 7B,H) but similar concentrations of butyrate (FIGS. 7C,I) and propionate (FIGS. 7D, J) compared to samples with high Bacteroidetes or Lachnospiraceae abundance.

While all taxonomic clusters had detectable concentrations of cholic acid, a primary BA, the highest concentrations were detected in Enterococcus dominated samples, likely reflecting the ability of bile salt hydrolase (BSH) expressing Enterococci to deconjugate glycocholic and taurocholic acid (FIG. 7M). Conjugated primary BA concentrations were lower (FIGS. 7E,K, L) and secondary (FIGS. 7F,N,O) and modified secondary BA (FIGS. 7G,P,R) concentrations were higher in samples with high levels of obligate anaerobes. Bifidobacteria-expanded samples had higher primary BA concentrations and lower secondary BA levels compared to Bacteroidetes- and Lachnospiraceae-dominated samples, suggesting Bifidobacteria provide increased BSH activity and/or reduced efficiency of primary to secondary BA conversion. However, compared to samples dominated by other taxa (e.g. Enterococcus, Proteobacteria, or Lactobacillus), Bifidobacteria-dominated samples were relatively adept at the full range of BA metabolism, including generation of secondary and modified secondary BA. The results indicate that a subset of patients with liver disease have expanded populations of Bifidobacteria in their GI tract, resulting in increased acetate concentrations and enhanced bile salt deconjugation while retaining modest ability to generate secondary BA.

### Lactulose Promotes Gut Bifidobacteria in Liver Disease

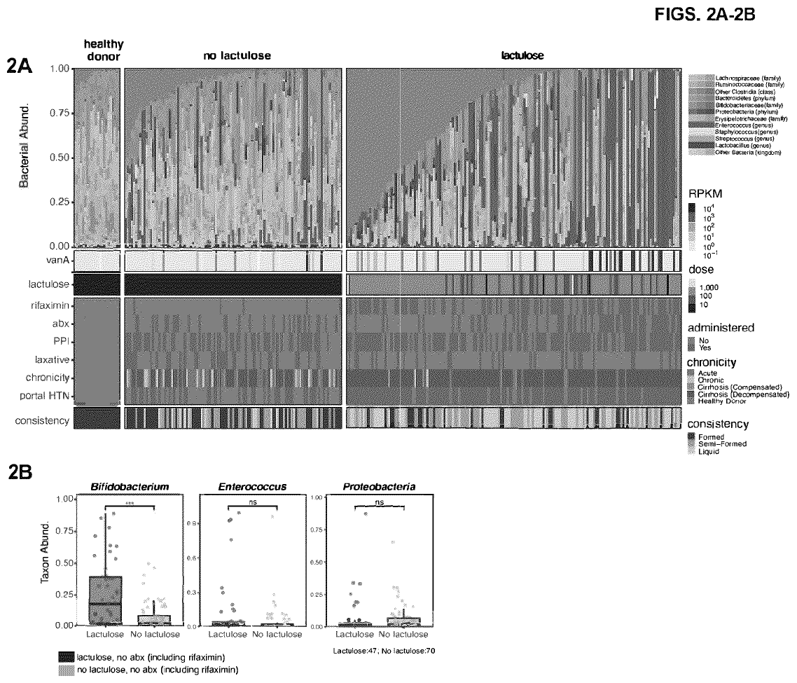

Lactulose is reported to have prebiotic activity that may impact microbiome compositions, but its impact on specific bacterial species and their production of metabolites remains poorly defined. To test whether lactulose is a driver of Bifidobacteria expansion in liver disease patients, the inventors ranked one fecal sample from each of the 262 patients by Bifidobacteria abundance and stratified patients by whether or not they had received lactulose within 7 days prior to sample collection. Fecal samples from lactulose treated patients were taxonomically diverse, with 45.2% having ≥10% Bifidobacteria abundance (FIG. 2A; range: 0.0-94.7%, mean: 22.2%, stdev: 28.4%). In contrast, fecal samples from patients not treated with lactulose had significantly lower Bifidobacteria abundances (FIG. 2A; range: 0.0-50.1%, mean: 6.8%, stdev: 10.4%, p_{adj}=0.009). High abundances (≥10%) of potential pathobionts including Enterobacteriaceae and Enterococcus were detected in lactulose treated and untreated patients (FIG. 2A). Enterococcus expansion was restricted to fecal samples with low Bifidobacteria abundance and was associated with the presence of the vancomycin-resistance gene vanA, most strikingly in samples from lactulose treated patients (FIG. 2A). These results suggest that lactulose-mediated Bifidobacteria expansion may limit intestinal Vancomycin-resistant Enterococcus (VRE) expansion. Treatment with broad-spectrum antibiotics likely reduces the density of Bifidobacterium species, potentially enabling VRE to benefit from lactulose treatment (FIG. 2A)^{25,41}. In support of this hypothesis, logistic regression demonstrates that lactulose administration positively associates with both Bifidobacteria and Enterococcus abundance (Table S1). Conversely, broad spectrum antibiotic administration negatively associates with Bifidobacteria abundance but positively associates with Enterococcus abundance (Table S1). Although the osmotic laxative effect of lactulose has been suggested to impact fecal microbiome compositions, the inventors did not detect an association between stool consistency and Bifidobacteria abundance (FIG. 2A, Table S1).

To exclude the potentially dramatic impact of antibiotics on lactulose-associated Bifidobacterium expansion, the inventors assessed the relative abundance of select taxa in samples obtained from patients who had not received antibiotics in the preceding 7 days (FIG. 2B). In this antibiotic-naïve cohort, Bifidobacteria density was significantly increased in fecal samples obtained from lactulose-treated patients while Enterococcus and Proteobacteria densities were similar between groups (FIG. 2B).

### Lactulose Promotes Bioactive Metabolites Production

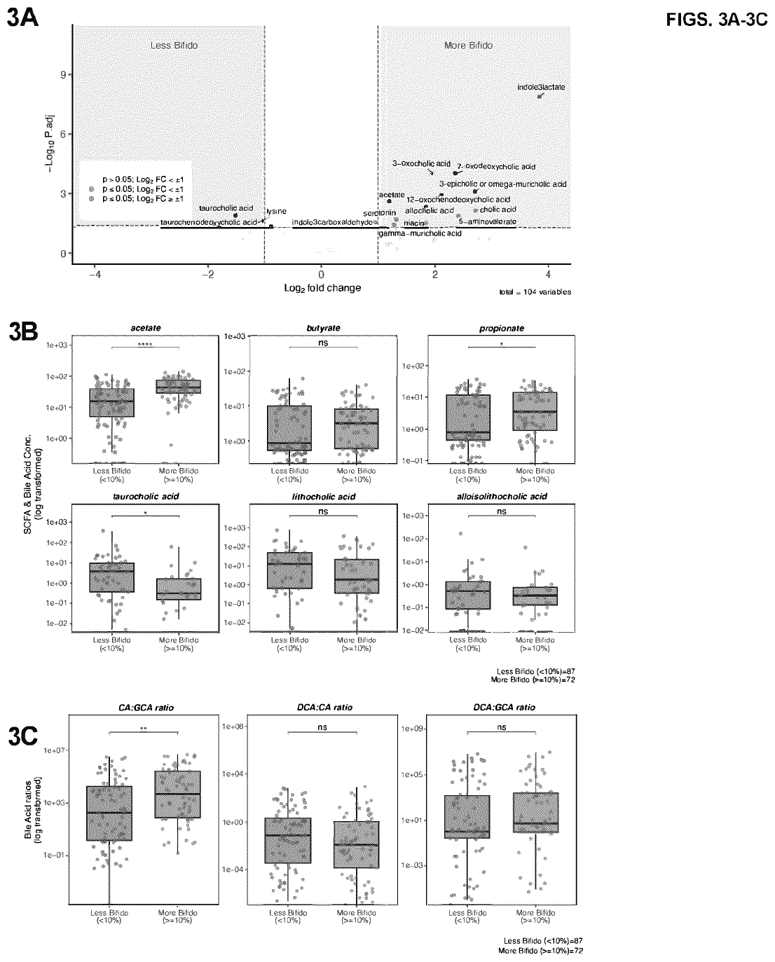

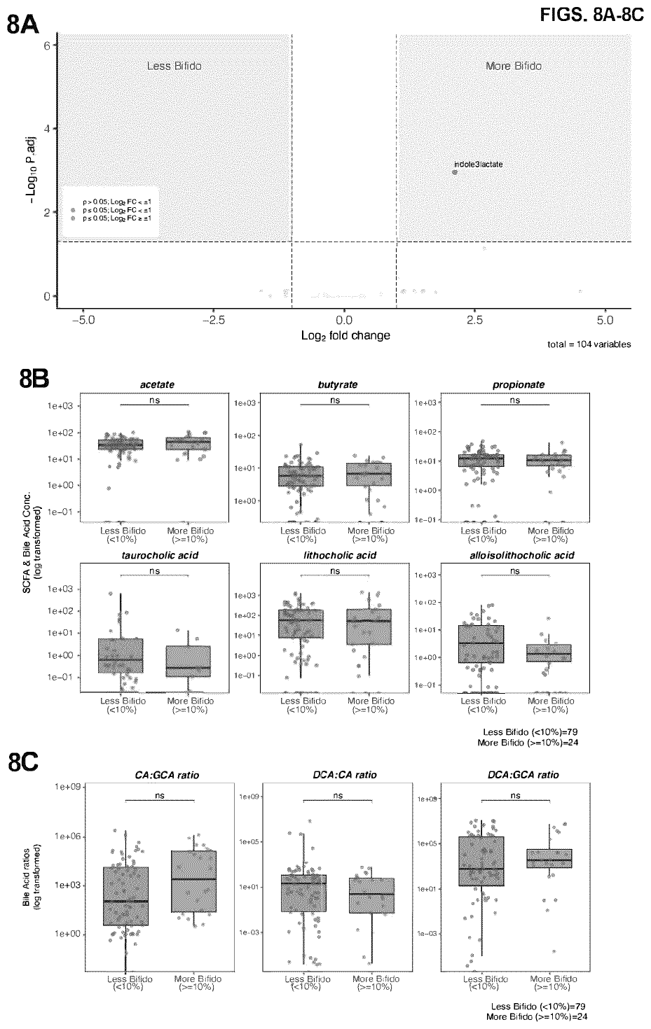

Bifidobacteria expansion, defined as relative abundance ≥10%, was associated with marked changes in fecal metabolite profiles in fecal samples from patients receiving lactulose (FIG. 3, 8). In patients with Bifidobacteria expansion, the fecal metabolome was characterized by increased fatty acid concentrations (including acetate), reduced conjugated primary BA concentrations (taurocholic and taurochenodeoxycholic acids), increased secondary and modified secondary BA (3-oxo-cholic acid, 7-oxo-deoxycholic), and indole metabolites (indole-3-lactic acid and to a lesser extent, indole-3-carboxaldehyde) (FIG. 3A,B). Consistent with a known property of Bifidobacteria to hydrolyze conjugated primary BA, ratios of unconjugated to conjugated primary BA were substantially higher in fecal samples with Bifidobacteria expansion (FIG. 3C). In patients not receiving lactulose, fecal samples with ≥10% Bifidobacteria had only modestly increased indole-3-lactic acid levels (FIG. 8). These results suggest that lactulose impacts the metabolic activity of the gut microbiome in liver disease patients (FIGS. 3 and 8).

Lactulose Enhances B. longum-Mediated VRE Inhibition

Many fecal samples obtained from lactulose-treated patients had high abundances of pathobionts belonging to Enterobacteriaceae family (range: 0.0-98.8%, mean: 7.9%, median: 0.9%, stdev: 17.7%) and the Enterococcus genus (range: 0.0-100%, mean: 19.4%, median: 0.9%, stdev: 34.4%), with higher densities than those seen in fecal samples taken from patients not receiving lactulose (Proteobacteria mean: 5.3%, median: 1.3%; Enterococcus mean: 5.0%, median: 0.05%). While high abundances of Proteobacteria species were detected across the range of Bifidobacteria abundances, Enterococcus domination was detected more commonly in fecal samples with reduced to absent Bifidobacteria abundance (FIG. 2A, 4A).

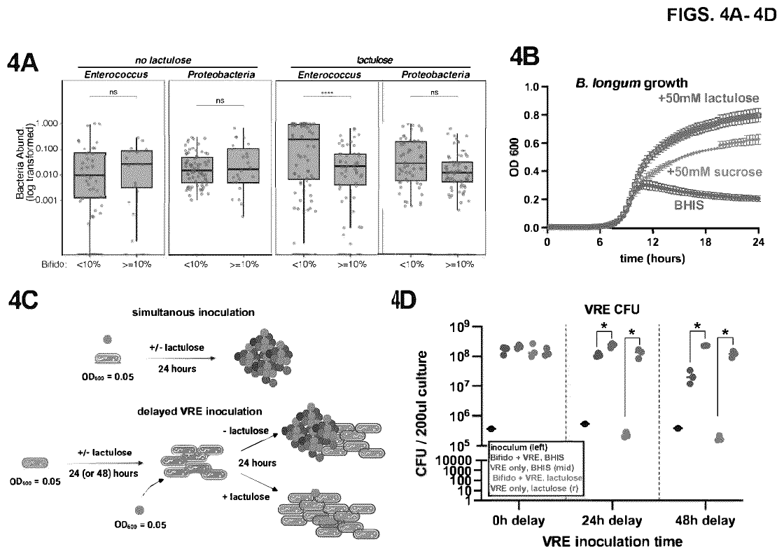

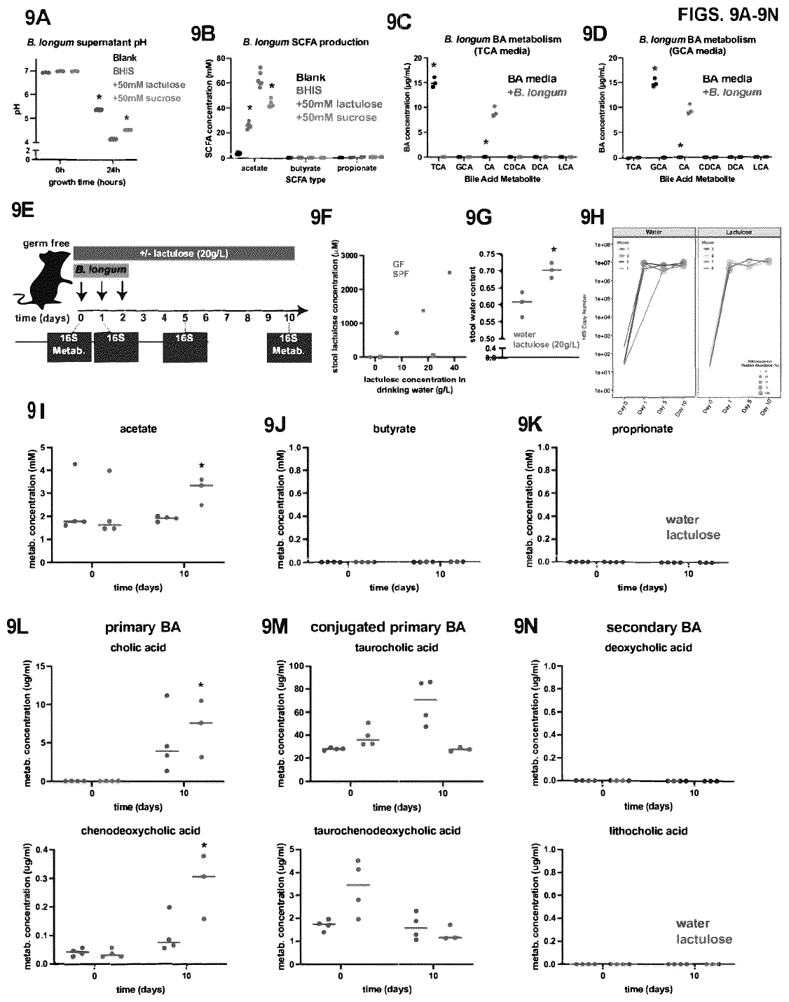

To investigate the inverse relationship between Bifidobacteria and VRE abundance, the inventors cultured and whole genome sequenced a healthy donor-derived Bifidobacterium longum strain that encodes genes required for lactulose metabolism and acetate production^{42,43.} B. longum culture growth was augmented by lactulose and to a lesser extent, sucrose (FIG. 4B). Lactulose-induced growth was accompanied by media acidification and acetate production (FIG. 9A,B). B. longum monocultures efficiently deconjugated taurocholic and glycocholic acid but, as expected, did not convert cholic acid to deoxycholic acid (FIG. 9C,D).

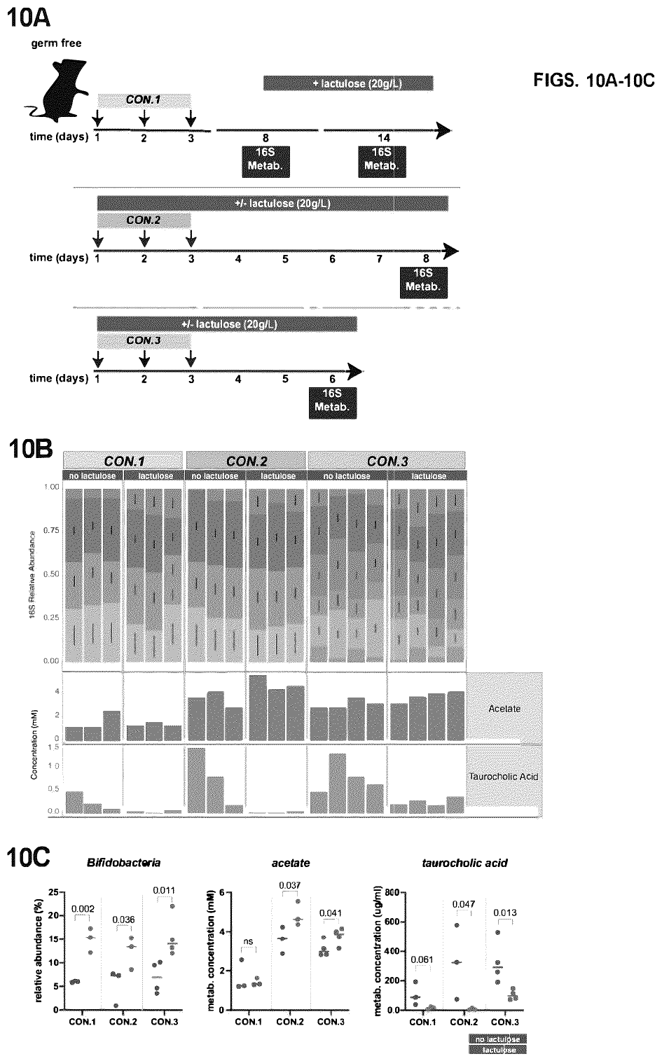

The inventors next colonized germ-free (GF) mice with B. longum and treated mice with or without lactulose (FIG. 9E). Lactulose was detectable in the stool of GF mice and led to increased stool water content (FIG. 9F,G). B. longum efficiently colonized and persisted in the intestines of gnotobiotic mice independent of lactulose (FIG. 9H). B. longum-colonized mice had measurable fecal acetate levels but did not generate butyrate or propionate (FIG. 9I-K). Lactulose treatment for 10 days resulted in elevated acetate and unconjugated primary BA concentrations and reduced concentrations of conjugated BA concentrations in B. longum colonized mice (FIGS. 9I,L,M). The inventors next colonized GF mice with more complex, 8-11 strain consortia that contain B. longum (FIG. 10 and Table S2). With all consortia, lactulose administration increased the relative abundance of B. longum (FIG. 10B,C). Lactulose reduced fecal taurocholic acid concentrations in mice colonized with all three consortia, potentially a result of enhanced B. longum-mediated BA deconjugation (FIG. 10B,C). Lactulose administration only modestly increased fecal acetate concentrations, possibly resulting from acetate consumption by other members of the administered consortia (FIG. 10B,C).

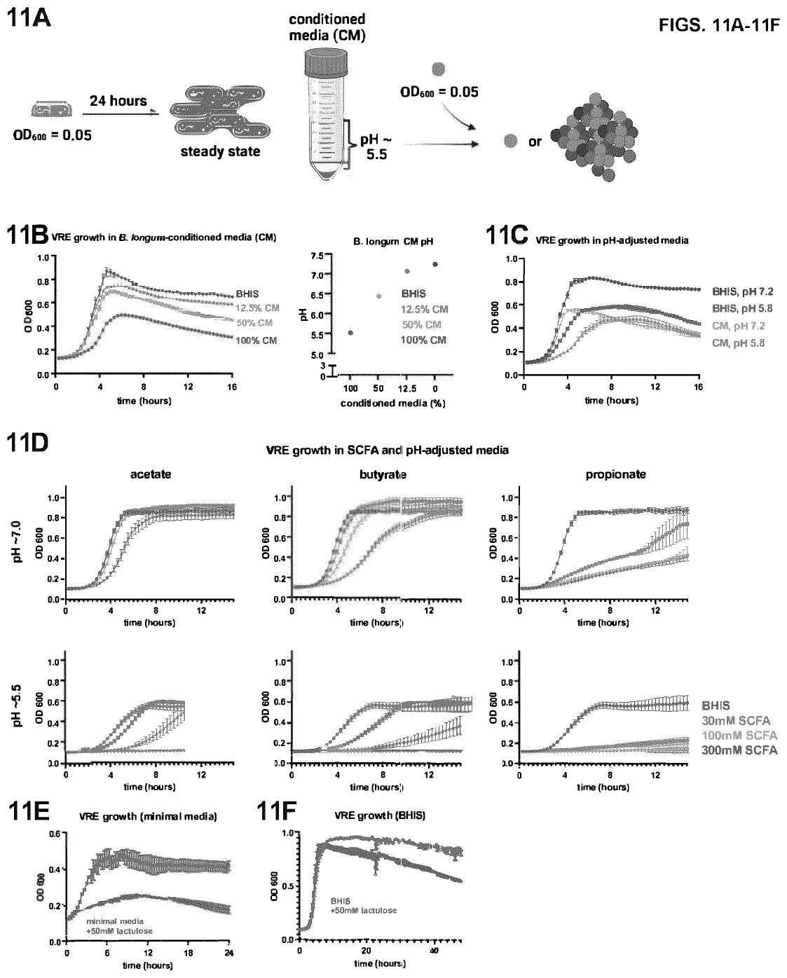

To test whether B. longum inhibits VRE growth and to determine the impact of lactulose administration, the inventors performed co-culture assays. Simultaneous inoculation of B. longum and VRE into media did not reduce VRE growth, but pre-culture of B. longum for 24 h or 48 h fully inhibited VRE growth in the presence of lactulose and only partially inhibited VRE growth in the absence of lactulose (FIG. 4C-D). Serial dilutions of B. longum culture supernatant with fresh media sequentially reduced the inhibitory effect on VRE growth (FIG. 11A, B). Alkalination of B. longum supernatant pH from 5.8 to 7.2 restored VRE's initial log phase growth but did not enable VRE to reach the maximal OD₆₀₀ obtained in fresh, neutral pH media (FIG. 11C). While lactulose modestly augments VRE growth, VRE growth is inhibited by addition of SCFA to the media, and inhibition is augmented at lower media pH (FIG. 11D-F). These results suggest that B. longum inhibits VRE growth through parallel mechanisms that include media acidification, SCFA production, and, to a lesser extent, nutrient deprivation.

Bifidobacteria Correlates with Decreased Infection Risk

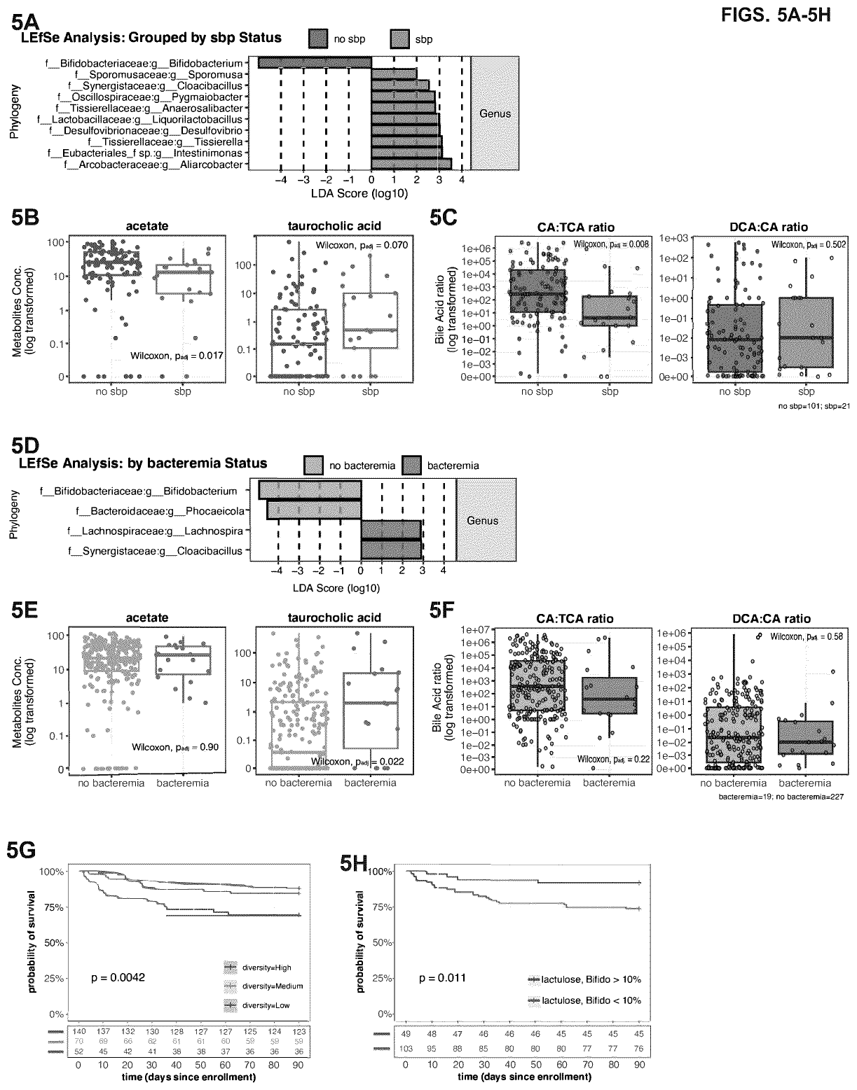

To determine whether lactulose-mediated Bifidobacteria expansion and associated metabolite changes are associated with clinical benefits, the inventors correlated microbiome compositional and metabolomic data with development of common infections in cirrhosis (FIGS. 5, 12, and 13).

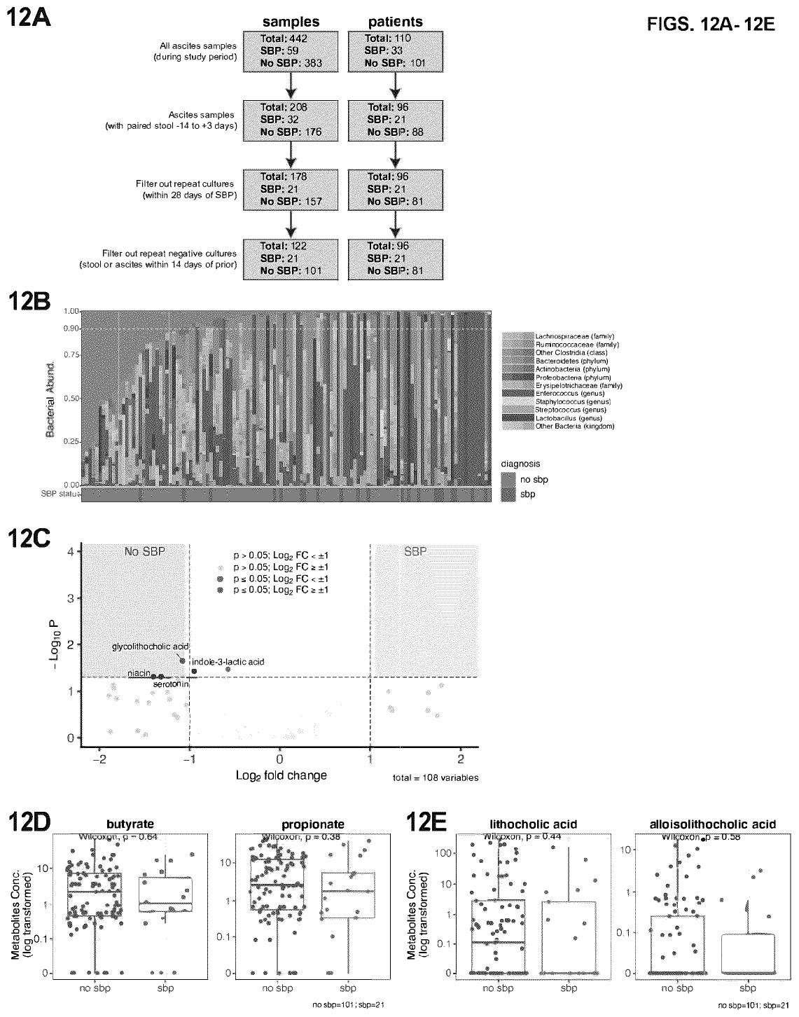

The inventors identified 122 ascites samples with near concurrent fecal samples, among which there were 21 diagnoses of SBP (FIG. 5A, 12A,B, Table S3). LEfSe analysis demonstrated that Bifidobacteria are associated with resistance to SBP (FIG. 5A). The inventors found that 1 of 21 patients (4.8%) with ≥10% Bifidobacteria frequency was diagnosed with SBP while 20 of 81 patients (24.7%) with <10% Bifidobacteria developed SBP. Upon correcting for MELD-Na at the time of ascitic fluid sampling and concurrent PPI use, Bifidobacteria expansion remained an independent predictor of remaining SBP-free (OR 0.09, 95% CI 0.00-0.54, p value 0.03, Tables S4, S5). Qualitative metabolomic analyses demonstrated that glycolithocholic acid, serotonin, and niacin were significantly associated with being SBP-free (FIG. 12C). The Bifidobacteria-generated metabolite indole-3-lactic acid was also increased in fecal samples from SBP-free patients, albeit to a lesser extent (p<0.05, log 2 fold-change 0.95, FIG. 12C). Quantitative metabolomics demonstrated that acetate was significantly higher in SBP-free fecal samples and taurocholic acid was higher, but not statistically significantly, in fecal samples from patients with SBP (FIG. 5B). Consistent with the BA-modifying properties of Bifidobacteria, samples from patients without SBP efficiently convert conjugated to unconjugated primary BA (CA:TCA ratio) but have similar capacity to convert primary to secondary BA (FIG. 5C).

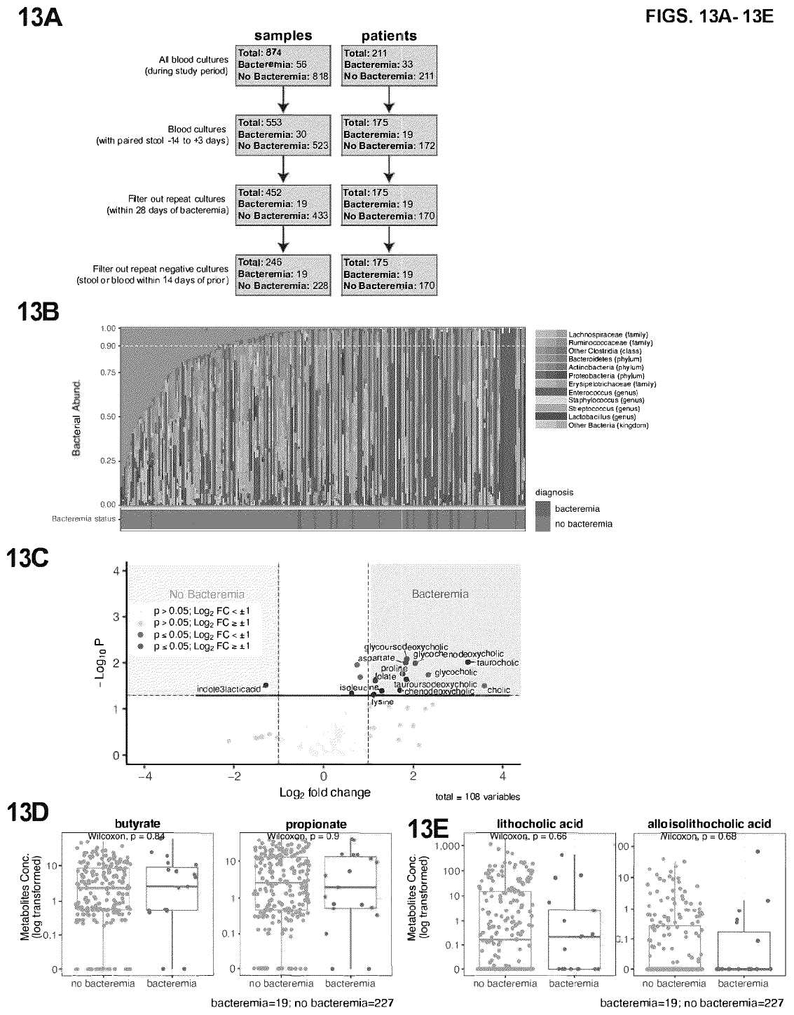

The inventors next paired 246 blood cultures with adjacent fecal samples for analysis and identified 19 diagnoses of bacteremia (FIG. 5D, 13A,B, Table S6). LEfSe analysis demonstrated that Bifidobacteria were associated with remaining bacteremia-free (FIG. 5D). There was 1 diagnosis of bacteremia associated with the 59 (1.7%) samples with ≥10% Bifidobacteria and 18 of 187 samples (9.6%) with <10% Bifidobacteria (OR: 0.16, 95% CI: 0.015 to 0.96). When correcting for MELD-Na at the time of blood culture and concurrent PPI, the odds ratio remained 0.16 with CI not including 1. However, likely due to the small number of events (only 19 bacteremia diagnoses), the p-value was 0.07 (Table S7). Qualitative metabolomics demonstrated that conjugated primary BA and primary BA were all significantly increased in bacteremia-associated samples whereas higher levels of the Bifidobacteria-derived indole-3-lactic acid were associated with sterile cultures (FIG. 13C). Targeted quantitative metabolomics confirmed that samples from bacteremic patients had increased taurocholic acid concentrations (FIG. 5E) but only modestly reduced ratio of unconjugated to conjugated BA ratio (FIG. 5F). These data demonstrate an association between Bifidobacteria expansion and protection from SBP and bacteremia and support the hypothesis that acetate production and BA deconjugation contribute to protection against some of the most common infectious complications of advanced liver disease.

Bifidobacteria Expansion Associates with Improved Survival

The inventors next assessed 90-day survival in patients stratified by either initial sample alpha-diversity (FIG. 5G) or lactulose exposure and Bifidobacteria expansion (FIG. 5H). Patients with low initial sample alpha-diversity had significantly reduced 90-day survival compared to those with medium or high initial sample alpha-diversity (FIG. 5G). Compared to patients who received lactulose but did not expand Bifidobacteria, patients with lactulose-associated Bifidobacteria expansion had significantly improved overall 90-day survival as assessed by Kaplan-Meier analysis (FIG. 5H). While many patient characteristics between the groups were similar, patients with low alpha-diversity and those who received lactulose but did not expand Bifidobacteria had other clinical parameters that portend a poor prognosis (Tables E1 and E2). While the unadjusted Kaplan-Meir analyses revealed significant differences between groups, when adjusting for multiple clinical parameters that could affect mortality with Cox proportional hazard models, the p-values of 0.26 (alpha-diversity) and 0.12 (Bifidobacteria expansion) fall short of the traditional 0.05 threshold despite hazard ratios of 1.52 (low alpha-diversity) and 0.42 (Bifidobacteria expansion) (Tables S9, S10). While microbiome diversity and the degree of Bifidobacterium expansion inversely correlate the risk of mortality, the lack of independence from clinical parameters that predict mortality is likely attributable to the intensity of interventions, including antibiotic treatment, that patients receive as the severity of CLD increases (Tables E1 and E2).

### Bifidobacteria Metabolizes Lactulose to Optimize Gut Metabolites and Prevent Systemic Infection in Liver Disease Patients.

Lactulose has been used to treat HE for over 50 years⁴⁴. Despite widespread use, lactulose's mechanism of action has remained incompletely defined^{7,15}. While lactulose is postulated to decrease ammonia absorption by decreasing bowel transit time and gut lumen acidification, its role in altering the microbiome has been largely unexplored. The inventors demonstrate that in patients with liver disease, lactulose leads to marked expansion of Bifidobacteria species in the absence of broad-spectrum antibiotics. This produces a distinct gut microbiome taxonomic and metabolic profile that is associated with exclusion of antibiotic-resistant pathobionts. Bifidobacteria inhibit in vitro growth of antibiotic-resistant Enterococcal species, which is augmented by lactulose. The findings suggest a protective role of lactulose-mediated Bifidobacteria expansion in patients with liver disease. Consistent with this, Bifidobacteria expansion and associated metabolites associate with reduced incidence of infection and improved 90-day survival.

In addition to treating precipitating factors (e.g. infection), lactulose has replaced dietary modification, antibiotics, and laxatives as first line treatment for HE. All HE therapies are aimed at reducing gut ammonia production and absorption, and the initial reports of lactulose use for HE concluded that this is due to luminal acidification^{44,45}. It has also been assumed that, similar to healthy subjects and experimental models^{12,46,47}, lactulose exhibits a prebiotic effect, but this has not been consistently shown in liver disease^{10,13,44,48,49}. The initial trial of lactulose was unable to detect changes in fecal bacteria⁴⁴; however, this study was limited by culture-based techniques. More recent studies characterizing the microbiomes of cirrhotic patients did not reveal Bifidobacteria expansion in patients treated with lactulose^{9,13}, likely because 16S rRNA sequencing platforms often do not amplify Bifidobacterium genes⁵⁰. Similarly, lactulose withdrawal demonstrated only slight decreases in Faecalibacterium species using sequencing techniques¹⁰. The inventors avoided 16S rRNA amplification bias by metagenomically sequencing fecal samples and demonstrate marked expansion of Bifidobacteria in a large cohort of cirrhotic patients receiving lactulose.

Bifidobacterium species are dominant fecal microbes in breast fed infants and are considered a prototypical health-promoting bacterium⁵¹. Bifidobacterium species imprint the human immune system⁵², are associated with decreased atopy and autoimmune diseases⁵³, and administration decreases rates of necrotizing enterocolitis in preterm infants⁵⁴. In adults, fecal Bifidobacteria modulate anti-tumor immunity and enhance immunotherapy efficacy in humans with melanoma and synergize with immunotherapies to reduce melanoma growth in mouse models^{55,56}. Bifidobacteria also produce acetate and lactic acid, which antagonize pathogens to reduce the incidence of enteric infections in infants. While Bifidobacteria do not provide colonization resistance to pathogenic E. coli in a murine model, specific acetate-producing strains limit systemic disease via acetate-mediated promotion of epithelial integrity and immune surveillance⁴². Gut acidification, mucosal barrier enhancement, and immunomodulatory effects of Bifidobacteria may all benefit patients with liver disease, who are immunocompromised and at high risk of enteric pathogen colonization, growth and dissemination.

While Bifidobacteria attenuate liver disease progression in rodents^{57,58}, the study suggests that Bifidobacteria may reduce complications of liver disease and enhance approaches for supportive care. The inventors report an association between Bifidobacteria expansion and reduced incidence of infection and prolonged survival. This beneficial association has multiple plausible explanations that may be driven by distinct metabolite profiles, including increased acetate levels and enhanced hydrolysis of conjugated primary BA. Similar metabolite profiles appear to be protective against both SBP and bacteremia. SCFA production has been linked to gut colonization resistance of common gram-negative enteric pathogens⁵⁹. Similarly, Bifidobacteria expansion was associated with reduced abundance of the common gram-positive pathogen, VRE in the patients, and inhibited VRE growth in vitro, likely via both media acidification and SCFA production. Bifidobacteria-mediated VRE growth inhibition was significantly enhanced by lactulose, which also significantly increased acetate production and acidification. While colonization and expansion are important initial steps in infection development, SCFAs also limit pathogen translocation by enhancing both epithelial integrity and mucosal immune function^{29,30,42,60}. Moreover, Bifidobacteria are associated with decreased intestinal permeability in patients with alcohol dependence, which is common in the cohort⁶¹. In the study, Bifidobacteria-expanded samples also had higher levels of indole-3-carboxaldehyde, which activates the aryl hydrocarbon receptor to increase mucosal IL-22 production and maintain reactivity towards pathogens.

Patients with liver disease are commonly treated with broad spectrum antibiotics with resultant dysbiosis. Infections precipitate hepatic decompensation and contribute to morbidity and mortality^{62,63}. The gut microbiome has been implicated in the development of infections in many disease states^{25,64}, including cirrhosis⁶⁵. A recent study used untargeted serum metabolites and fecal 16S data to predict infections in cirrhosis⁶⁶; however, many identified metabolites were not microbially derived and did not substantially improve the ability to predict infection beyond standard clinical metrics⁶⁶. Antibiotics, while highly effective for treatment and prophylaxis against common infections^{67,68}, are associated with increasing antibiotic resistance genes in the gut microbiomes and subsequent poor outcomes⁶⁹⁻⁷².

While associated with improved outcomes in the population, a Bifidobacteria-expanded microbiome is not reflective of healthy adult microbiome. Acetate prevents gut pathogen dissemination in a murine model, but butyrate is better studied with regard to epithelial barrier function and mucosal immune function^{29,30,32,42}. Secondary BA are considered markers of a “healthy” gut microbiome but have also been implicated in prevention of enteric infections⁶⁴ and reduced intestinal inflammation⁸⁵.

In conclusion, the inventors report that lactulose-mediated gut Bifidobacteria expansion can be associated with a distinct fecal microbiome compositional and metabolic profile, reduced antibiotic-resistant pathobiont burden, and improved clinical outcomes in hospitalized patients with liver disease, including reduced incidence of infection and prolonged survival.

### Example 2—Materials and Methods for Aspects Herein

### Study Design

This was a prospective cohort study of consecutive hospitalized adult hepatology patients at a single institution from April 2021 to April 2022. Inclusion criteria were age ≥18 years, ability to provide informed consent (either themselves or by proxy if unable to provide consent), and being treated on the hepatology consult service. Subjects who were younger than 18 years, unable to provide consent, had prior solid organ transplant, or a prior colectomy were excluded. Patients were enrolled as soon as possible upon hospital admission, most within 48 hours. Samples were obtained under a protocol approved by the Institutional Review Board at the University of Chicago (IRB21-0327). Written informed consent was obtained from all participants or their surrogate decision makers. Participants were not compensated to take part in this observational study.

### Specimen Collection and Storage

After enrollment, an order for stool collection was placed in the electronic medical record. Stool samples were collected by the clinical nursing teams on inpatient wards and intensive care units. After collection, samples were immediately sent to the microbiology lab through the pneumatic tubing system and stored at +4° C. until collection by the research team. The research team collected freshly obtained refrigerated samples twice daily, and all samples were all aliquoted and stored at −80° C. within 24 hours of sample production. If able to provide additional samples, fecal samples were collected approximately every 2 days during hospitalization. Samples were collected in a similar manner on re-hospitalization up until 1 year post-enrollment or until death or transplant. Samples remained stored at −80° C. until they were processed for metagenomics and metabolomics.

### Clinical Data Collection:

Upon enrollment, all patients were given a unique patient ID that was linked to their unique medical record numbers (MRNs). The unique ID was stored in a RedCap database along with prior to admission medications and disease characteristics, which were obtained by a combination of patient/family recollection and verification with medical records when available. After enrollment, clinical data (including inpatient medication information and laboratory values) was all gathered from the Center for Research Informatics (CRI: https://cri.uchicago.edu/) at the University of Chicago. This is a clinical data warehouse that contains all medication administration records (MAR), laboratory values, and additional clinical parameters linked to each MRN. The CRI database and RedCap data were merged through MRNs, and select records were verified by chart review to ensure accuracy. All survival data was verified with chart review. There were no discrepancies identified between CRI database and the electronic medical record.

### Infectious Data:

Infectious data was obtained through the CRI data warehouse and verified with manual chart review. Infections were defined using standard clinical criteria. That is, ascitic fluid infections without an evident intraabdominal source (e.g. bowel perforation) were all considered spontaneous bacterial peritonitis (SBP) and were diagnosed with the standard clinical definition of either polymorphonuclear cells (PMN) of 250 cells/mm³ or greater or a positive ascites culture^{67,68}. Cultures growing common contaminants (i.e. components of skin flora) were considered negative if PMN were <250 cells/mm³ and the clinical team did not treat for SBP. Blood stream infection (i.e. bacteremia) was defined as having a blood culture with bacterial or fungal growth. Skin contaminants were again excluded (i.e. considered “negative” cultures) if the clinical team did not treat for bacteremia. Clinical samples were paired with the closest stool sample that was within 14 days prior to the ascitic sample or 3 days after the ascitic sample. If a clinical sample indicated infection, all subsequent samples for the subsequent 28 days were excluded from analysis to minimize observing the effects of directed antibiotic therapy. If an initial ascitic fluid sample or blood culture was negative, subsequent clinical samples were excluded for 14 days. Flow diagrams for this approach are shown in Figure S11.

### Metagenomic Analyses

Fecal samples underwent shotgun DNA sequencing. After undergoing mechanical disruptions with a bead beater (BioSpec Product), samples were further purified with QIAamp mini spin columns (Qiagen). Purified DNA was quantified with a Qubit 2.0 fluorometer and sequenced on the Illumina HiSeq platform, producing around 7 to 8 million PE reads per sample with read length of 150 bp. Adapters were trimmed off from the raw reads, and their quality were assessed and controlled using Trimmomatic (v.0.39) 87, then human genome were removed by kneaddata (v0.7.10, https://github.com/biobakery/kneaddata). Taxonomy was profiled using metaphlan4 using the resultant high-quality reads⁸⁸. Microbial reads then were assembled using MEGAHIT called genes (v1.2.9)⁸⁹, are by prodigal (https://github.com/hyattpd/Prodigal). In addition, high-quality reads are queried against genes of interest, such as virulence factors, cazymes, and antibiotic resistance genes, using DIAMOND (v2.0.4) 90, and hits are filtered with threshold >80% identity, >80% protein coverage, then abundance is tabulated into counts per million or reads per million mapped reads (RPKM).

Alpha-diversity of fecal samples was estimated using the Inverse Simpson Index, while beta diversity was assessed by using taxumap (https://github.com/jsevo/taxumap). The inventors applied Uniform Manifold Approximation and Project (UMAP) on taxonomy profiles on the 847 Liver Disease samples using a slightly modified approach, taxUMAP⁹¹. Number of neighbors was 375, while no custom weighting of the aggregations of taxon abundances was applied. Each sample is represented by a single point and colored by most abundant/dominant taxon as indicated. Samples with no taxon >5% relative abundance were considered to have no most abundant taxon and were labeled as “other.” Metagenomic information is publicly available on NCBI under BioProject ID PRJNA912122 (liver disease cohort) and BioProject ID PRJNA838648 (healthy donor cohort).

### Mouse Fecal DNA Isolation

DNA was extracted using the QIAamp PowerFecal Pro DNA kit (Qiagen). Prior to extraction, samples were subjected to mechanical disruption using a bead beating method. Briefly, samples were suspended in a bead tube (Qiagen) along with lysis buffer and loaded on a bead mill homogenizer (Fisherbrand). Samples were then centrifuged, and supernatant was resuspended in a reagent that effectively removed inhibitors. DNA was then purified routinely using a spin column filter membrane and quantified using Qubit.

### 16S Sequencing

16S sequencing was performed for murine studies in which known bacterial strains that were previously whole genome sequenced were given to ex-germ-free mice. V4-V5 region within 16S rRNA gene was amplified using universal bacterial primers-563F (5′-nnnnnnnn-NNNNNNNNNNNN-AYTGGGYDTAAA-GNG-3′) (5′-nnnnnnnn-NNNNNNNNNNNN-CCGTCAATTYHT-TTRAGT-3′), where ‘N’ represents the barcodes, ‘n’ are additional nucleotides added to offset primer sequencing. Approximately ˜412 bp region amplicons were then purified using a spin column-based method (Minelute, Qiagen), quantified, and pooled at equimolar concentrations. Illumina sequencing-compatible Unique Dual Index (UDI) adapters were ligated onto the pools using the QIAseq 1-step amplicon library kit (Qiagen). Library QC was performed using Qubit and Tapestation and sequenced on Illumina MiSeq platform to generate 2×250 bp reads.

16S qPCR

16S qPCR was performed for murine studies in which ex-germ-free mice were monocolonized with Bifidobacteria given that all mice had 100% relative abundance of Bifidobacteria. Extracted DNA was diluted to 20 ng/ul to ensure concentrations fell within measurable range. Degenerate primers were diluted to 5.5 mM concentration. Primer sequences are as follows: 563F (5′-AYTGGGYDTAAAGNG-3′) and 926Rb (5′-CCGTCAATTYHTTTRAGT-3′). Standard curves were generated using linearized TOPO pcr2.1TA vector (containing V4-V5 region of the 16S rRNA gene) transformed into DH5α competent bacterial cells. Five-fold serial dilution was performed on the purified plasmid from 108 to 103 copies/μl per tube. PCR products were detected using PowerTrack SYBR Green Master Mix (A46109). qPCR was run on QuantStudio 6 Pro (Applied Biosystems) with the following cycling conditions: 95° C. for 10 min, followed by 40 cycles of 95° C. for 30 s, 52° C. for 30 s, and 72° C. for 1 min. Copy numbers for samples were calculated using the Design and Analysis v2 software.

### Metabolomic Analyses

Short chain fatty acids (SCFA, i.e. butyrate, acetate, propionate, and succinate) were derivatized with pentafluorobenzyl bromide (PFBBr) and analyzed via negative ion collision induced-gas chromatography-mass spectrometry ([-]CI-GC-MS, Agilent 8890) 92. Eight bile acids (BA, i.e. primary: cholic acid; conjugated primary: glycocholic acid, taurocholic acid; secondary: deoxycholic acid, lithocholic acid [LCA], isodeoxycholic acid; modified secondary: alloisolithocholic acid [alloisoLCA] and 3-oxolithocholic acid [3-oxoLCA]) were quantified (μg/mL) by negative mode liquid chromatography-electrospray ionization-quadrupole time-of-flight-MS ([-]LC-ESI-QTOF-MS, Agilent 6546). Eleven indole metabolites were quantified by UPLC-QqQ LC-MS. Eighty-five additional compounds were relatively quantified using normalized peak areas relative to internal standards. Data analysis was performed using MassHunter Quantitative Analysis software (version B.10, Agilent Technologies) and confirmed by comparison to authentic standards. Quantitative fecal metabolomic information paired to fecal metagenomic information is publicly available on NCBI under BioProject ID PRJNA912122 (liver disease cohort) and BioProject ID PRJNA838648 (healthy donor cohort). Raw data files are publicly available on MetaboLights project ID MTBLS7046 (both liver disease and healthy donor cohorts).

### Bacterial Culture

The Bifidobacteria longum strains MSK.11.12 and DFI.2.45 were previously derived from two distinct healthy donor stool samples and whole genome sequenced (BioSample ID: SAMN19731851 and SAMN22167409). The vancomycin resistant Enterococcus faecium (VRE) strain used in this study was obtained from ATCC (strain 700221). Both bacterial strains were grown in anaerobic conditions in Brain-heart infusion broth (BHI broth, BD 237500). The pH was adjusted to 7.0 with NaOH. Media was supplemented with lactulose (Thermo Scientific, J60160-22) or sucrose (Fisher BP220-1) where indicated. For growth in media supplemented with short chain fatty acids, media was supplemented with either sodium butyrate (Sigma 303410), sodium succinate (Sigma S2378) or sodium acetate (Sigma S5636). For studies of bile acid metabolism, 10 μg/mL of conjugated primary bile acid, either glycocholic acid (Sigma T4009) or taurocholic acid (EMD Millipore, 360512), was added to the media. All growth curves were obtained in anaerobic culture conditions at 37° C. on a BioTek EPOCH2 microplate reader with BioTek Gen 5 3.11 software. Growth curves were analyzed in GraphPad Prism Version 9.4.0. Lactulose concentrations in were measured using the EnzyChrom™ Lactulose Assay Kit (ELTL-100).

### Mouse Studies

All mouse studies were approved by The University of Chicago Institutional Animal Care and Use Committee (IACUC, Protocol 72599). For germ-free studies, 6-18 week-old male and female C57BL/6 mice were used. Mice were initially obtained from The Jackson Laboratory and subsequently bred and raised in a germ-free isolator. After removal from the germ-free isolator, mice were handled in a sterile manner and individually housed in sealed negative pressure (BCU) isolators. Mice were fed an ad libitum diet of Teklad Global 18% Protein Rodent Diet (Sterilizable) (2018S/2018SC). Mice were treated with either regular sterile water or sterile water supplemented with filter sterilized lactulose at a final concentration of 20 g lactulose/L of water in the timing indicated in Figures S6 and S7. For monocolonization, B. longum was grown to steady state in BHIS, pelleted, and resuspended in an equal volume of PBS, and previously germ-free mice were gavaged with 200 μL of a freshly prepared suspension on 3 consecutive days. For experiments with consortia, the consortia strains are as follows. CON.1 and CON.2: A. hadrus, B. longum, B. ovatus, C. comes, C. scindens, C. symbiosum, E. lenta, L. gasseri, P. distasonis, P. merdae, and R. gnavus. CON.3: A. caccae, B. longum, B. ovatus, C. scindens, C. symbiosum, E. lenta, P. distasonis, and P. vugatus. Details regarding strain ID and Biosample ID for each strain are given in Table S2. Each consortia strain was grown to early steady state and normalized to OD_{600=0.3}. Consortia strains were grown in BHIS, Wilkins-Chalgren (Fisher), de Man, Regosa, and Sharpe (MRS) broth (Fisher), or modified Yeast Casitone Fatty Acids (YCFA) medium termed YTFA medium (recipe shown in Table S11). Stocks of the consortia were stored at −80° C. in 20% glycerol, 0.1% cysteine until ready for use, and previously germ-free mice were gavaged with 100 μL (CON.1 and CON.2) or 200 μl (CON.3) of the consortia for 3 consecutive days. Fecal pellets were collected at the indicated timepoints for 16S rRNA metagenomic analysis and targeted metabolomic analysis.

### Statistical Analyses

All statistical analyses were conducted using the R programming language (version 4.2.2). Adjusted p-values of the tests were considered to be statistically significant for all analyses conducted if p≤0.05. Continuous variables were compared between the groups using Wilcoxon rank-sum test (rstatix::wilcox_test) and multiple test correction were adjusted following the Benjamini-Hochberg method (stats:: p.adjust). Categorial variables were compared using Fisher's Exact test (stats::fisher.test). Linear regression (stats::lm) was used to estimate the response of an outcome (FIG. 3: Bifidobacteria and Enterococcus abundance) to multiple factors (FIG. 3: lactulose exposure, PPI use, antibiotics, and stool consistency). Results of linear regression are shown in Table S1. Logistic regression (stats::glm) was used to estimate the odds of multiple factors (FIG. 6: Bifidobacteria abundance, PPI use, MELD-Na score) affecting outcomes (FIG. 6: SBP and bacteremia). Results of logistic regression are shown in FIGS. 6B and 6F. Kaplan-Meier curves for survival were generated after stratifying for selected microbiome parameters including alpha-diversity (high, medium, and low, FIG. 61) and Bifidobacteria abundance after lactulose exposure (FIG. 61) (survival::Surv, survfit, ggsurvplot). A Cox proportional hazards regression model for mortality was used to estimate the effects of microbiome parameters (alpha-diversity and Bifidobacterium abundance after lactulose exposure) adjusting for known risk factors for mortality in liver disease (survival::coxph). Results of Cox proportional hazard testing are shown in Tables S7 and S8.

### Example 3—Tables Included in Aspects Herein

TABLE A1
Patient demographics and baseline disease characteristics of all patients that produced at least one stool sample. Patients
are stratified by liver disease chronicity at the time of consent. Clinically significant portal hypertension was defined
by hepatic venous pressure gradient (HVPG) ≥ 10 mmHg, characteristic imaging (enlarged portal vein, intraabdominal
varices, splenomegaly, or ascites), or clinical features (ascites, varices, or hepatic encephalopathy).

| | | | | | | Cirrhosis | | Cirrhosis | | |
|---|---|---|---|---|---|---|---|---|---|---|
| | | Acute | | Chronic | | (Compensated) | | (Decompensated) | | |
| | n | 35 | | 18 | | 13 | | 196 | | p |
| Basic | Age (median | 57.30 | [34.00, 67.25] | 60.20 | [48.57, 64.60] | 63.10 | [54.90, 69.70] | 58.05 | [47.62, 65.93] | 0.413 |
| Demographics | [IQR]) | | | | | | | | | |
| | Sex = Male (%) | 12 | (34.3) | 10 | (55.6) | 9 | (69.2) | 123 | (62.8) | 0.014 |
| | Race (%) | | | | | | | | | 0.894 |
| | African American | 10 | (28.6) | 8 | (44.4) | 4 | (30.8) | 64 | (32.7) | |
| | Asian/Pacific Islander | 3 | (8.6) | 1 | (5.6) | 1 | (7.7) | 7 | (3.6) | |
| | Caucasian | 17 | (48.6) | 6 | (33.3) | 7 | (53.8) | 95 | (48.5) | |
| | Hispanic | 5 | (14.3) | 2 | (11.1) | 1 | (7.7) | 26 | (13.3) | |
| | Other | 0 | (0.0) | 1 | (5.6) | 0 | (0.0) | 4 | (2.0) | |
| | BMI (median [IQR]) | 25.28 | [21.61, 28.50] | 26.61 | [21.84, 32.33] | 30.67 | [26.75, 34.17] | 27.99 | [23.62, 33.04] | 0.059 |
| Disease | Liver Disease | | | | | | | | | <0.001 |
| Etiology | Etiology (%) | | | | | | | | | |
| | AIH | 5 | (14.3) | 0 | (0.0) | 1 | (7.7) | 6 | (3.1) | |
| | Alcohol | 5 | (14.3) | 1 | (5.6) | 3 | (23.1) | 124 | (63.3) | |
| | Checkpoint | 4 | (11.4) | 0 | (0.0) | 0 | (0.0) | 1 | (0.5) | |
| | Blockade Hepatitis | | | | | | | | | |
| | DIL | 6 | (17.1) | 0 | (0.0) | 0 | (0.0) | 0 | (0.0) | |
| | HBV | 0 | (0.0) | 0 | (0.0) | 0 | (0.0) | 3 | (1.5) | |
| | HCV | 0 | (0.0) | 0 | (0.0) | 1 | (7.7) | 15 | (7.7) | |
| | Metabolic | 0 | (0.0) | 0 | (0.0) | 0 | (0.0) | 4 | (2.0) | |
| | NASH | 0 | (0.0) | 1 | (5.6) | 3 | (23.1) | 23 | (11.7) | |
| | Other | 7 | (20.0) | 3 | (16.7) | 2 | (15.4) | 6 | (3.1) | |
| | PBC | 0 | (0.0) | 0 | (0.0) | 0 | (0.0) | 3 | (1.5) | |
| | PSC | 0 | (0.0) | 1 | (5.6) | 0 | (0.0) | 4 | (2.0) | |
| | Vascular | 8 | (22.9) | 12 | (66.7) | 3 | (23.1) | 7 | (3.6) | |
| Disease Severity | MELD-Na at consent | 10.93 | [5.30, 15.47] | 10.40 | [2.56, 20.19] | 13.06 | [4.74, 19.98] | 21.30 | [13.46, 31.92] | <0.001 |
| | (median [IQR]) | | | | | | | | | |
| | Clinically Significant | 2 | (5.7) | 2 | (11.1) | 5 | (38.5) | 174 | (88.8) | <0.001 |
| | Portal HTN (%) | | | | | | | | | |
| | HCC (%) | 1 | (2.9) | 1 | (5.6) | 3 | (23.1) | 22 | (11.2) | 0.171 |
| | NACSELD-ACLF at | 0 | (0.0) | 0 | (0.0) | 0 | (0.0) | 29 | (14.8) | 0.012 |
| | Consent (%) | | | | | | | | | |
| | Hepatic | 2 | (5.7) | 1 | (5.6) | 0 | (0.0) | 56 | (28.6) | 0.001 |
| | Encephalopathy (%) | | | | | | | | | |
| | Shock (%) | 2 | (5.7) | 3 | (16.7) | 1 | (7.7) | 26 | (13.3) | 0.54 |
| | Respiratory | 1 | (2.9) | 1 | (5.6) | 1 | (7.7) | 25 | (12.8) | 0.29 |
| | Failure (%) | | | | | | | | | |
| | Renal Failure (%) | 1 | (2.9) | 0 | (0.0) | 0 | (0.0) | 24 | (12.2) | 0.081 |
| Medications | Lactulose | | | | | | | | | |
| and Diet | PTA (%) | 1 | (2.9) | 2 | (11.1) | 0 | (0.0) | 85 | (43.6) | <0.001 |
| | Inpatient (%) | 7 | (20.0) | 5 | (27.8) | 5 | (38.5) | 148 | (75.9) | <0.001 |
| | Rifaximin | | | | | | | | | |
| | PTA (%) | 0 | (0.0) | 1 | (5.6) | 0 | (0.0) | 60 | (30.8) | <0.001 |
| | Inpatient (%) | 5 | (14.3) | 1 | (5.6) | 2 | (15.4) | 103 | (52.8) | <0.001 |
| | Broad Antibiotics | | | | | | | | | |
| | (non-rifaximin) | | | | | | | | | |
| | PTA (%) | 5 | (14.3) | 3 | (16.7) | 1 | (7.7) | 52 | (26.7) | 0.169 |
| | Inpatient (%) | 10 | (28.6) | 4 | (22.2) | 5 | (38.5) | 80 | (41.0) | 0.263 |
| | Diet at time | | | | | | | | | 0.52 |
| | of consent (%) | | | | | | | | | |
| | Diet Order | 31 | (88.6) | 15 | (83.3) | 13 | (100.0) | 155 | (79.5) | |
| | NPO | 4 | (11.4) | 3 | (16.7) | 0 | (0.0) | 37 | (19.0) | |
| | Tube Feeds | 0 | (0.0) | 0 | (0.0) | 0 | (0.0) | 3 | (1.5) | |
| Disease | SBP (%) | 0 | (0.0) | 1 | (5.6) | 0 | (0.0) | 20 | (10.2) | 0.133 |
| Complications | Bacteremia (%) | 2 | (5.7) | 2 | (11.1) | 0 | (0.0) | 15 | (7.7) | 0.663 |
| | 90-day Survival (%) | 33 | (94.3) | 18 | (100.0) | 12 | (92.3) | 155 | (79.1) | 0.02 |

EXTENDED TABLE 1: Patient demographics and baseline disease characteristics stratified by initial sample alpha-diversity. Samples were grouped into high, medium, and low alpha-diversity based on tertiles of inverse Simpson levels derived from the full cohort of 847 samples.

TABLE E1

| | | High | | Medium | | Low | | p value |
|---|---|---|---|---|---|---|---|---|
| Demographics | n | 140 | | 70 | | 52 | | |
| | Age (median [IQR]) | 59.15 | [44.10, 66.93] | 60.00 | [48.85, 66.05] | 55.90 | [49.02, 65.30] | 0.646 |
| | Sex = Male (%) | 81 | (57.9) | 41 | (58.6) | 32 | (61.5) | 0.899 |
| | Race (%) | | | | | | | 0.278 |
| | African American | 48 | (34.3) | 21 | (30.0) | 17 | (32.7) | |
| | Asian/Pacific Islander | 7 | (5.0) | 4 | (5.7) | 1 | (1.9) | |
| | Caucasian | 61 | (43.6) | 33 | (47.1) | 31 | (59.6) | |
| | Hispanic | 20 | (14.3) | 12 | (17.1) | 2 | (3.8) | |
| | Other | 4 | (2.9) | 0 | (0.0) | 1 | (1.9) | |
| | BMI (median [IQR]) | 27.35 | [23.95, 32.95] | 29.24 | [22.95, 32.77] | 26.07 | [22.90, 32.45] | 0.425 |
| Disease Chonicity | Disease Chronicity (%) | | | | | | | <0.001 |
| | Acute | 23 | (16.4) | 9 | (12.9) | 3 | (5.8) | |
| | Chronic | 16 | (11.4) | 2 | (2.9) | 0 | (0.0) | |
| | Cirrhosis (Compensated) | 12 | (8.6) | 0 | (0.0) | 1 | (1.9) | |
| | Cirrhosis (Decompensated) | 89 | (63.6) | 59 | (84.3) | 48 | (92.3) | |
| Etiology | Primary Disease Etiology (%) | | | | | | | 0.138 |
| | AIH | 10 | (7.1) | 1 | (1.4) | 1 | (1.9) | |
| | Alcohol | 59 | (42.1) | 36 | (51.4) | 38 | (73.1) | |
| | Checkpoint Blockade Hepatitis | 5 | (3.6) | 0 | (0.0) | 0 | (0.0) | |
| | DILI | 4 | (2.9) | 2 | (2.9) | 0 | (0.0) | |
| | HBV | 1 | (0.7) | 2 | (2.9) | 0 | (0.0) | |
| | HCV | 9 | (6.4) | 5 | (7.1) | 2 | (3.8) | |
| | Metabolic | 2 | (1.4) | 1 | (1.4) | 1 | (1.9) | |
| | NASH | 13 | (9.3) | 11 | (15.7) | 3 | (5.8) | |
| | Other | 12 | (8.6) | 4 | (5.7) | 2 | (3.8) | |
| | PBC | 3 | (2.1) | 0 | (0.0) | 0 | (0.0) | |
| | PSC | 3 | (2.1) | 1 | (1.4) | 1 | (1.9) | |
| | Vascular | 19 | (13.6) | 7 | (10.0) | 4 | (7.7) | |
| Severity | MELD-Na (median [IQR]) | 15.25 | [8.55, 24.20] | 17.79 | [10.63, 27.29] | 30.86 | [21.22, 37.15] | <0.001 |
| | Clinically Significant Portal HTN (%) | 82 | (58.6) | 55 | (78.6) | 46 | (88.5) | <0.001 |
| | ACLF-NACSELD (%) | 8 | (5.7) | 7 | (10.0) | 14 | (26.9) | <0.001 |
| | HE (%) | 15 | (10.7) | 22 | (31.4) | 22 | (42.3) | <0.001 |
| | Shock (%) | 15 | (10.7) | 7 | (10.0) | 10 | (19.2) | 0.223 |
| | Respiratory Failure (%) | 11 | (7.9) | 9 | (12.9) | 8 | (15.4) | 0.256 |
| | Renal Failure (%) | 6 | (4.3) | 6 | (8.6) | 13 | (25.0) | <0.001 |
| Medications | Lactulose | | | | | | | |
| | PTA | 28 | (20.0) | 35 | (50.0) | 25 | (49.0) | <0.001 |
| | Inpatient | 66 | (47.1) | 55 | (78.6) | 44 | (86.3) | <0.001 |
| | Rifaximin | | | | | | | |
| | PTA | 19 | (13.6) | 26 | (37.1) | 16 | (31.4) | <0.001 |
| | Inpatient | 32 | (22.9) | 45 | (64.3) | 34 | (66.7) | <0.001 |
| | Antibiotics | | | | | | | |
| | PTA | 19 | (13.6) | 18 | (25.7) | 24 | (47.1) | <0.001 |
| | Inpatient | 43 | (30.7) | 26 | (37.1) | 30 | (58.8) | 0.002 |
| Complications | HCC (%) | 18 | (12.9) | 8 | (11.4) | 1 | (1.9) | 0.081 |
| | SBP (any time) (%) | 10 | (7.1) | 5 | (7.1) | 6 | (11.5) | 0.579 |
| | Bacteremia (any time) (%) | 9 | (6.4) | 6 | (8.6) | 4 | (7.7) | 0.845 |
| | Death (within 90 days) (%) | 17 | (12.1) | 11 | (15.7) | 16 | (30.8) | 0.009 |
| Sample Characteristics | inverse Simpson (median [IQR]) | 13.83 | [9.33, 18.65] | 3.87 | [3.24, 4.83] | 1.27 | [1.02, 1.59] | <0.001 |

EXTENDED TABLE 2: Patient demographics and baseline disease characteristics stratified by initial sample Bifidobacteria expansion in response to lactulose.

TABLE E2

| | | lactulose, bifido <10% | | lactulose, bifido >10% | | p value |
|---|---|---|---|---|---|---|
| Demographics | n | 103 | | 49 | | |
| | Age (median [IQR]) | 57.80 | [49.30, 64.65] | 62.50 | [51.30, 67.30] | 0.178 |
| | Sex = Male (%) | 68 | (66.0) | 24 | (49.0) | 0.067 |
| | Race (%) | | | | | 0.576 |
| | African American | 30 | (29.1) | 16 | (32.7) | |
| | Asian/Pacific Islander | 3 | (2.9) | 4 | (8.2) | |
| | Caucasian | 57 | (55.3) | 22 | (44.9) | |
| | Hispanic | 11 | (10.7) | 6 | (12.2) | |
| | Other | 2 | (1.9) | 1 | (2.0) | |
| | BMI (median [IQR]) | 28.38 | [23.68, 33.45] | 28.00 | [23.20, 33.20] | 0.628 |
| Disease Chonicity | Disease Chronicity (%) | | | | | 0.512 |
| | Acute | 7 | (6.8) | 1 | (2.0) | |
| | Chronic | 1 | (1.0) | 1 | (2.0) | |
| | Cirrhosis (Compensated) | 2 | (1.9) | 2 | (4.1) | |
| | Cirrhosis (Decompensated) | 93 | (90.3) | 45 | (91.8) | |
| Etiology | Primary Disease Etiology (%) | | | | | 0.155 |
| | AIH | 1 | (1.0) | 1 | (2.0) | |
| | Alcohol | 69 | (67.0) | 25 | (51.0) | |
| | Checkpoint Blockade Hepatitis | 1 | (1.0) | 0 | (0.0) | |
| | DILI | 4 | (3.9) | 0 | (0.0) | |
| | HBV | 1 | (1.0) | 1 | (2.0) | |
| | HCV | 4 | (3.9) | 5 | (10.2) | |
| | Metabolic | 2 | (1.9) | 0 | (0.0) | |
| | NASH | 10 | (9.7) | 10 | (20.4) | |
| | Other | 3 | (2.9) | 4 | (8.2) | |
| | PBC | 0 | (0.0) | 1 | (2.0) | |
| | PSC | 2 | (1.9) | 0 | (0.0) | |
| | Vascular | 6 | (5.8) | 2 | (4.1) | |
| Severity | MELD-Na (median [IQR]) | 25.58 | [16.24, 35.18] | 18.85 | [12.26, 25.75] | 0.002 |
| | Clinically Significant Portal HTN (%) | 89 | (86.4) | 43 | (87.8) | 1 |
| | ACLF-NACSELD (%) | 24 | (23.3) | 0 | (0.0) | 0.001 |
| | HE (%) | 44 | (42.7) | 12 | (24.5) | 0.046 |
| | Shock (%) | 21 | (20.4) | 1 | (2.0) | 0.006 |
| | Respiratory Failure (%) | 17 | (16.5) | 4 | (8.2) | 0.254 |
| | Renal Failure (%) | 19 | (18.4) | 0 | (0.0) | 0.003 |
| Medications | Lactulose | | | | | |
| | PTA | 59 | (57.8) | 29 | (59.2) | 1 |
| | Inpatient | 95 | (93.1) | 44 | (89.8) | 0.697 |
| | Rifaximin | | | | | |
| | PTA | 35 | (34.3) | 21 | (42.9) | 0.402 |
| | Inpatient | 72 | (70.6) | 31 | (63.3) | 0.473 |
| | Antibiotics | | | | | |
| | PTA | 38 | (37.3) | 8 | (16.3) | 0.015 |
| | Inpatient | 57 | (55.9) | 9 | (18.4) | <0.001 |
| Complications | HCC (%) | 3 | (2.9) | 9 | (18.4) | 0.003 |
| | SBP (any time) (%) | 14 | (13.6) | 2 | (4.1) | 0.133 |
| | Bacteremia (any time) (%) | 5 | (4.9) | 3 | (6.1) | 1 |
| | Death (within 90 days) (%) | 27 | (26.2) | 4 | (8.2) | 0.018 |

TABLE S1

| | Estimate | Std Error | Statistic | p-value |
|---|---|---|---|---|
| Bifidobacteria | | | | |
| (Intercept) | 0.10 | 0.03 | 3.14 | 1.88E−03 |
| Lactulose | 0.17 | 0.03 | 5.65 | 4.35E−08 |
| PPI | 0.05 | 0.03 | 1.61 | 0.11 |
| Stool Consistency (Liquid) | −0.04 | 0.04 | −0.97 | 0.33 |
| Stool Consistency | −0.02 | 0.03 | −0.56 | 0.58 |
| (Semi-Formed) | | | | |
| Antibiotics | −0.15 | 0.03 | −5.02 | 9.69E−07 |
| Enterococcus | | | | |
| (Intercept) | −0.01 | 0.04 | −0.32 | 0.75 |
| Lactulose | 0.11 | 0.04 | 2.77 | 6.01E−03 |
| PPI | 0.05 | 0.04 | 1.39 | 0.17 |
| Stool Consistency (Liquid) | 0.05 | 0.05 | 1.06 | 0.29 |
| Stool Consistency | −0.02 | 0.04 | −0.50 | 0.62 |
| (Semi-Formed) | | | | |
| Antibiotics | 0.13 | 0.04 | 3.62 | 3.50E−04 |

TABLE S2

| Bacterial | | | | | | |
|---|---|---|---|---|---|---|
| Species | Strain ID | BioSample ID | Media | CON. 1 | CON. 2 | CON. 3 |
| A. caccae | DFI.7.76 | SAMN28944557 | BHIS | − | − | + |
| A. hadrus | DFI.4.30 | SAMN22187445 | BHIS | + | + | − |
| B. longum | DFI.2.45 | SAMN22167409 | BHIS | + | + | − |
| B. longum | DFI.4.163 | SAMN24725995 | BHIS | − | − | + |
| B. ovatus | MSK.22.29 | SAMN19732130 | YTFA | + | + | − |
| B. ovatus | MSK.18.37 | SAMN19731980 | YTFA | − | − | + |
| C. comes | DFI 3.84 | SAMN22167436 | BHIS | + | + | − |
| C. scindens | DFI.4.158 | Not available | BHIS | + | − | − |
| C. scindens | SL.1.22 | SAMN22167568 | BHIS | − | + | + |
| C. symbiosum | DFI.5.64 | SAMN22187467 | BHIS | + | + | + |
| E. lenta | MSK.14.3 | SAMN19731872 | Wilkins-Chalgren | + | + | − |
| E. lenta | MSK.5.72 | Not available | Wilkins-Chalgren | − | − | + |
| L. gasseri | DFI.2.88 | SAMN28944486 | MRS | + | + | − |
| P. distasonis | DFI.5.23 | SAMN28944503 | YTFA | + | + | + |
| P. merdae | DFI.4.73 | SAMN28944501 | YTRA | + | + | − |
| P. vulgatus | DFI.4.81 | SAMN19731837 | YTFA | − | − | + |
| R. gnavus | MSK.15.77 | SAMN14067611 | BHIS | + | + | − |

TABLE S3

| | | Stool-to-Ascites | Bifido- | Entero- | Proteo- | Escherichia | |
|---|---|---|---|---|---|---|---|
| PMN | Culture | Time (days) | bacterium | coccus | bacteria | coli | Klebsiella |
| 0 | gram positive cocci | [symbol] | 0.05 | 37.16 | 18.27 | [symbol] 7.99 | 0.22 |
| 1139 | No growth | −11 | 0.00 | 95.32 | 0.00 | 0.00 | 0.00 |
| 7165.08 | No growth | [symbol] | 0.00 | 0.00 | 1.08 | 0.00 | 0.20 |
| 861.96 | No growth | [symbol] | 0.12 | 0.00 | 1.99 | 0.00 | 0.00 |
| 24950 | E. faecium, | [symbol] | 0.60 | 5.68 | 6.38 | 0.00 | 1. [symbol] 4 |
| | C. freundii | | | | | | |
| 4.895 | C. jeikeium, gram | 0 | 23.74 | 2.71 | 0.95 | 0.00 | 0.02 |
| | positive bacilli | | | | | | |
| 1105.1 | No growth | 0 | 0.13 | 86.18 | 1.71 | 1.70 | 0.00 |
| 1004.08 | No growth | −1 | 5.69 | 0.91 | 0.48 | 0.24 | 0.00 |
| 278 | No growth | −2 | 0.00 | 0.53 | 1.95 | 0.58 | 0.1 [symbol] |
| 22847.5 | No growth | 0 | 0.00 | 0.00 | 0.00 | 0.00 | 0.02 |
| 1015.585 | No growth | 2 | 0.00 | 0.00 | 0.00 | 0.00 | 0.00 |
| 149.35 | E. coli | 3 | 0.01 | 0.00 | 0.00 | 0.00 | 0.00 |
| 1585.08 | E. coli [ESBL] | 0 | 0.00 | 1.10 | 33.58 | 27.36 | 0.00 |
| 1085.4 | No growth | 2 | 5.02 | 2.34 | [symbol] .37 | 15.31 | 0.00 |
| 7147.56 | P. multocida | 3 | 0.00 | 0.74 | 0.97 | 0.00 | 0.00 |
| 1790.52 | No growth | −3 | 0.00 | 89.20 | 0.00 | 0.00 | 0.00 |
| 1700.16 | C. albicans | 2 | 0.00 | 0.01 | 0. [symbol] | 0.33 | 0.00 |
| 1349.24 | No growth | 0 | 0.00 | 0.02 | 8.97 | 8.75 | 0.00 |
| 77.665 | C. striatum | 2 | 0.00 | 100.00 | 0.00 | 0.00 | 0.00 |
| 790.72 | E. faecium | [symbol] | 0.78 | 13.92 | 89.34 | 68.77 | 0.27 |
| 315.5 | No growth | −4 | 0.00 | 1.88 | 3.03 | 0.00 | 0.00 |

[symbol]

indicates data missing or illegible when filed

TABLE S4

| | | | |
|---|---|---|---|
| | no sbp, | sbp, | |
| Characteristic | N = 101¹ | N = 21¹ | p-value² |

| | | | | | |
|---|---|---|---|---|---|
| PPI | 59 | (58%) | 10 | (40%) | 0.4 |
| MELD-Na | | | | | 0.002 |
| Median | 25 | (16, 31) | 32 | (27,39) | |
| (IQR) | | | | | |
| Mean | 24 | (7, 40) | 31 | (12, 40) | |
| (Range) | | | | | |
| Lactulose | | | | | 0.8 |
| Lactulose | 80 | (79%) | 16 | (76%) | |
| No lactulose | 21 | (21%) | 5 | (24%) | |
| Bifidobacteria | | | | | 0.02 |
| Abundace | | | | | |
| >10% | 28 | (28%) | 1 | (4.7%) | |
| <10% | 73 | (72%) | 20 | (95.2%) | |
| Enterococcus | | | | | 0.6 |
| Abundance | | | | | |
| >10% | 35 | (35%) | 6 | (29%) | |
| <10% | 66 | (65%) | 15 | (71%) | |

¹n (%)
²Pearson's Chi-squared test; Wilcoxon rank sum test, Fisher's

TABLE S5
SBP

| | OR (95% CI) | p-value |
|---|---|---|
| (Intercept) | 0.03 (0.00-0.16) | 3.04E−04 |
| PPI | 0.32 (0.10-0.97) | 0.05 |
| MELD-Na | 1.11 (1.04-1.20) | 0 |
| Bifido >10% | 0.09 (0.00-0.54) | 0.03 |

TABLE S6

| | Stool-to-blood | Bifido- | Entero- | Proteo- | Escherichia | |
|---|---|---|---|---|---|---|
| Blood Culture Growth | time (days) | bacterium | coccus | bacteria | coli | Klebsiella |
| E. faecium | 2 | 42.93 | 11.32 | 2.28 | 0.00 | 2.25 |
| [symbol] -hemolytic-streptococci | 50 | 0.00 | 0.00 | 1.08 | 0.00 | 0.20 |
| E. faecium | −5 | 0.00 | 97.10 | 0.00 | 0.00 | 0.00 |
| P. aeruginosa | 0 | 0.00 | 99.09 | 0.13 | 0.00 | 0.00 |
| S. [symbol] , Gemella species | [symbol] | 0.00 | 91.80 | 0.23 | 0.00 | 0.00 |
| K. pneumoniae [ESBL] | −3 | 0.00 | 73.26 | 7.63 | 0.00 | 7.63 |
| P. aeruginosa | 3 | 0.00 | 0.00 | 0.00 | 0.00 | 0.00 |
| K. pneumoniae [ [symbol] ] | 2 | 0.00 | 0.00 | 0.00 | 0.00 | 0.00 |
| E. coli [ESBL] | −3 | 0.00 | 0.00 | 40.99 | 27.11 | 0.00 |
| gram positive bacilli | 2 | 0.00 | 10.45 | 31.97 | 45.90 | 0.19 |
| C. lusitaniae | −1 | 0.17 | 13.53 | 2.84 | 0.24 | 0.13 |
| S. aureus (MRSA) | 0 | 0.36 | 5.24 | 0. [symbol] | 0.87 | 0.00 |
| K. pneumoniae | 2 | 0.94 | 0.01 | 1.00 | 0.87 | 0.11 |
| Bacillus species, not anthracis | 0 | 0.06 | 0.43 | 0.00 | 0.00 | 0.00 |
| E. coli | 3 | 0.00 | 8.04 | 9.34 | 9.34 | 0.00 |
| E. cloacae | 3 | 0.13 | 0.00 | 1.62 | 1.02 | 0.02 |
| P. multocida, coagulase negative | 3 | 0.00 | 0.74 | 0.97 | 0.00 | 0.00 |
| Staphylococcus species | | | | | | |
| gram positive bacilli | 0 | 0.97 | 0.00 | 7.56 | 7.32 | 0.00 |
| C. [symbol] | 1 | 0.00 | 31.96 | 24. [symbol] 9 | 24.33 | 0.01 |

[symbol]

indicates data missing or illegible when filed

TABLE S7

| | | | |
|---|---|---|---|
| | bacteremia, | no bacteremia, | |
| Characteristic | N = 19¹ | N = 227¹ | p-value² |

| | | | | | |
|---|---|---|---|---|---|
| PPI | 7 | (37%) | 114 | (50%) | 0.3 |
| MELD-Na | | | | | 0.6 |
| Median | 18 | (13, 32) | 23 | (15, 31) | |
| (IQR) | | | | | |
| Mean | 22 | (2, 40) | 23 | (1, 40) | |
| (Range) | | | | | |
| Lactulose | | | | | 0.004 |
| Lactulose | 6 | (32%) | 140 | (65%) | |
| No lactulose | 13 | (68%) | 79 | (35%) | |
| Bifidobacteria | | | | | 0.05 |
| Abundance | | | | | |
| >10% | 1 | (5.3%) | 58 | (25.6%) | |
| <10% | 18 | (94.7%) | 169 | (74.4%) | |
| Enterococcus | | | | | 0.3 |
| Abundance | | | | | |
| >10% | 8 | (42%) | 70 | (33%) | |
| <10% | 11 | (58%) | 157 | (69%) | |

¹n (%)
²Pearson's Chi-squared test; Wilcoxon rank sum test; Fisher's

TABLE S8
Bacteremia

| | OR (95% CI) | p-value |
|---|---|---|
| (Intercept) | 0.17 (0.05-0.49) | 0 |
| PPI | 0.60 (0.21-1.58) | 0.31 |
| MELD-Na | 0.99 (0.95-1.03) | 0.6 |
| Bifido >10% | 0.16 (0.01-0.80) | 0.07 |

TABLE S9

| | | Std | | | Exp |
|---|---|---|---|---|---|
| | Estimate | Error | Statistic | p-value | (coef) |
| Low alpha-diversity | 0.42 | 0.37 | 1.12 | 0.26 | 1.52 |
| Medium alpha-diversiy | 0.02 | 0.39 | 0.05 | 0.96 | 1.02 |
| MELD-Na (enrollment) | 0.08 | 0.02 | 4.51 | 6.48E−06 | 1.08 |
| Age (enrollment) | 0.02 | 0.01 | 1.65 | 0.10 | 1.02 |
| Sex (male) | −0.15 | 0.32 | −0.49 | 0.63 | 0.86 |

TABLE S10

| | | Std | | | Exp |
|---|---|---|---|---|---|
| | Estimate | Error | Statistic | p-value | (coef) |
| Lactulose, Bifido >10% | −0.87 | 0.56 | −1.56 | 0.12 | 0.42 |
| MELD-Na (enrollment) | 0.09 | 0.02 | 4.11 | 3.98E−05 | 1.1 |
| Age (enrollment) | 0.04 | 0.02 | 2.2 | 0.03 | 1.04 |
| Sex (male) | −0.11 | 0.38 | −0.27 | 0.78 | 0.9 |

TABLE S11
YTFA Medium
To Autoclave

| | | |
|---|---|---|
| Tryptone (BD-Difco) | 10.0 | g |
| Yeast extract (BD-Difco) | 2.5 | g |
| NaHCO3 | 4.0 | g |
| Glucose | 5.0 | g |
| Maltose | 2.0 | g |
| Cellobiose | 2.0 | g |
| Mineral solution I | 150.0 | ml |
| Mineral solution II | 150.0 | ml |
| VFA mix | 6.2 | ml |
| Distilled water | 660.0 | ml |
| Adjust pH to 6.80 | 1.0 | g |

After Autoclaving

| | | |
|---|---|---|
| L-Cysteine-HCl•H2O | | |
| Hemin solution | 10.0 | ml |
| Vitamin solution I | 1.0 | ml |
| Vitamin solution II | 1.0 | ml |

Vitamin and Mineral Solotions
Mineral solution I:

| | | |
|---|---|---|
| K2HPO4 | 3.0 | g |
| Distilled water | 1.0 | L |

Mineral solution II:

| | | |
|---|---|---|
| KH2PO4 | 3.0 | g |
| (NH4)2SO4 | 6.0 | g |
| NaCl | 6.0 | g |
| MgSO4•7H2O | 0.6 | g |
| CaCl2•2H2O | 0.6 | g |
| Distilled water | 1.0 | L |

VFA mix:

| | | |
|---|---|---|
| Acetic acid | 17.0 | ml |
| Propionic acid | 6.0 | ml |
| n-Valeric acid | 1.0 | ml |
| iso-Valeric acid | 1.0 | ml |
| iso-Butyric acid | 1.0 | ml |

Hemin solution:

| | | |
|---|---|---|
| KOH | 0.28 | g |
| Ethanol | 25.0 | ml |
| Hemin | 0.1 | g |
| Adjust volume to 100 ml with distilled water. | | |

Vitamin solution I:

| | | |
|---|---|---|
| Biotin | 5.0 | mg |
| Vitamin B12 | 5.0 | mg |
| p-Aminobenzoic acid | 15.0 | mg |
| Folic acid | 25.0 | mg |
| Pyridoxine•HCl | 75.0 | mg |
| Distilled water | 500.0 | ml |

Vitamin solution II:

| | | |
|---|---|---|
| Thiamine•HCl | 25.0 | mg |
| Riboflavin | 25.0 | mg |
| Distilled water | 500.0 | ml |

### REFERENCES

The following references, to the extent that they provide exemplary procedural or other details supplementary to those set forth herein, are specifically incorporated herein by reference.
1. Moon, A. M., Singal, A. G. & Tapper, E. B. Contemporary Epidemiology of Chronic Liver Disease and Cirrhosis. Clin Gastroenterol H 18, 2650-2666 (2019).
2. Younossi, Z. M. et al. Epidemiology of chronic liver diseases in the USA in the past three decades. Gut 69, 564 (2020).
3. Termeie, O. et al. Alarming Trends: Mortality from Alcoholic Cirrhosis in the United States. Am J Medicine (2022) doi: 10.1016/j.amjmed.2022.05.015.
4. Collaborators, G. 2017 C. et al. The global, regional, and national burden of cirrhosis by cause in 195 countries and territories, 1990-2017: a systematic analysis for the Global Burden of Disease Study 2017. Lancet Gastroenterology Hepatology 5, 245-266 (2020).
5. Franchis, R. de et al. BAVENO VII-RENEWING CONSENSUS IN PORTAL HYPERTENSION Report of the Baveno VII Consensus Workshop: personalized care in portal hypertension. J Hepatol 76, 959-974 (2021).
6. Ge, P. S. & Runyon, B. A. Treatment of Patients with Cirrhosis. New Engl J Medicine 375, 767-777 (2016).
7. Vilstrup, H. et al. Hepatic encephalopathy in chronic liver disease: 2014 Practice Guideline by the American Association for the Study Of Liver Diseases and the European Association for the Study of the Liver. Hepatology 60, 715-735 (2014).
8. Riggio, O. et al. Effect of Lactitol and Lactulose Administration on the Fecal Flora in Cirrhotic Patients. J Clin Gastroenterol 12, 433-436 (1990).
9. Bajaj, J. S. et al. Altered profile of human gut microbiome is associated with cirrhosis and its complications. J Hepatol 60, 940-947 (2014).
10. Bajaj, J. S. et al. A longitudinal systems biology analysis of lactulose withdrawal in hepatic encephalopathy. Metab Brain Dis 27, 205-215 (2012).
11. Haemmerli, Peter, U. & Bircher, and J. Wrong Idea, Good Results (The Lactulose Story). New England Journal of Medcine (1969).
12. Ruszkowski, J. & Witkowski, J. M. Lactulose: Patient- and dose-dependent prebiotic properties in humans. Anaerobe 59, 100-106 (2019).
13. Wang, J. Y. et al. Lactulose Improves Cognition, Quality of Life and Gut Microbiota in Minimal Hepatic Encephalopathy: A Multi-Center, Randomized Controlled Trial. J. Dig. Dis. 20, 547-556 (2019).
14. Bajaj, J. S. et al. Nosocomial Infections Are Frequent and Negatively Impact Outcomes in Hospitalized Patients With Cirrhosis. Am J Gastroenterol 114, 1091-1100 (2019).
15. Gluud, L. L., Vilstrup, H. & Morgan, M. Y. Nonabsorbable disaccharides for hepatic encephalopathy: A systematic review and meta-analysis. Hepatology 64, 908-922 (2016).
16. Llopis, M. et al. Intestinal microbiota contributes to individual susceptibility to alcoholic liver disease. Gut 65, 830 (2016).
17. Duan, Y. et al. Bacteriophage targeting of gut bacterium attenuates alcoholic liver disease. Nature 575, 505-511 (2019).
18. Hermanson, J. B. et al. Dietary Cholesterol-Induced Gut Microbes Drive Nonalcoholic Fatty Liver Disease Pathogenesis in a Murine Model. FASEB (2022).
19. Qin, N. et al. Alterations of the human gut microbiome in liver cirrhosis. Nature 513, 59-64 (2014).
20. Dubinkina, V. B. et al. Links of gut microbiota composition with alcohol dependence syndrome and alcoholic liver disease. Microbiome 5, 141 (2017).
21. Caussy, C. et al. A gut microbiome signature for cirrhosis due to nonalcoholic fatty liver disease. Nat Commun 10, 1406 (2019).
22. Loomba, R. et al. Gut Microbiome-Based Metagenomic Signature for Non-invasive Detection of Advanced Fibrosis in Human Nonalcoholic Fatty Liver Disease. Cell Metab 25, 1054-1062.e5 (2017).
23. Bajaj, J. S. et al. Gut microbial RNA and DNA analysis predicts hospitalizations in cirrhosis. Jci Insight 3, e98019 (2018).
24. Ahluwalia, V. et al. Impaired Gut-Liver-Brain Axis in Patients with Cirrhosis. Sci Rep-uk 6, 26800 (2016).
25. Taur, Y. et al. Intestinal Domination and the Risk of Bacteremia in Patients Undergoing Allogeneic Hematopoietic Stem Cell Transplantation. Clin Infect Dis 55, 905-914 (2012).
26. Peled, J. U. et al. Microbiota as Predictor of Mortality in Allogeneic Hematopoietic-Cell Transplantation. New Engl J Med 382, 822-834 (2020).
27. Stutz, M. R. et al. Immunomodulatory fecal metabolites are associated with mortality in COVID-19 patients with respiratory failure. Nat Commun 13, 6615 (2022).
28. Roediger, W. E. W. Utilization of Nutrients by Isolated Epithelial Cells of the Rat Colon. Gastroenterology 83, 424-429 (1982).
29. Augeron, C. & Laboisse, C. L. Emergence of permanently differentiated cell clones in a human colonic cancer cell line in culture after treatment with sodium butyrate. Cancer Res 44, 3961-9 (1984).
30. Peng, L., Li, Z.-R., Green, R. S., Holzman, I. R. & Lin, J. Butyrate Enhances the Intestinal Barrier by Facilitating Tight Junction Assembly via Activation of AMP-Activated Protein Kinase in Caco-2 Cell Monolayers. J Nutrition 139, 1619-1625 (2009).
31. Hang, S. et al. Bile acid metabolites control Th17 and Treg cell differentiation. Nature 576, 143-148 (2019).
32. Arpaia, N. et al. Metabolites produced by commensal bacteria promote peripheral regulatory T cell generation. Nature 504, 451-455 (2013).
33. Mouzaki, M. et al. Bile Acids and Dysbiosis in Non-Alcoholic Fatty Liver Disease. Plos One 11, e0151829 (2016).
34. Kasai, Y. et al. Association of Serum and Fecal Bile Acid Patterns With Liver Fibrosis in Biopsy-Proven Nonalcoholic Fatty Liver Disease: An Observational Study. Clin Transl Gastroen 13, e00503 (2022).
35. Adams, L. A. et al. Bile acids associate with specific gut microbiota, low-level alcohol consumption and liver fibrosis in patients with non-alcoholic fatty liver disease. Liver Int 40, 1356-1365 (2020).
36. Leonhardt, J. et al. Circulating Bile Acids in Liver Failure Activate TGR5 and Induce Monocyte Dysfunction. Cell Mol Gastroenterology Hepatology 12, 25-40 (2021).
37. Alm, R., Carlson, J. & Eriksson, S. Fasting Serum Bile Acids in Liver Disease. Scand J Gastroentero 17, 213-218 (2010).
38. Ferslew, B. C. et al. Altered Bile Acid Metabolome in Patients with Nonalcoholic Steatohepatitis. Digest Dis Sci 60, 3318-3328 (2015).
39. O'Leary, J. G. et al. NACSELD acute-on-chronic liver failure (NACSELD-ACLF) score predicts 30-day survival in hospitalized patients with cirrhosis. Hepatology 67, 2367-2374 (2018).
40. Stoma, I. et al. Compositional Flux Within the Intestinal Microbiota and Risk for Bloodstream Infection With Gram-negative Bacteria. Clin Infect Dis 73, ciaa068 (2020).
41. Ubeda, C. et al. Vancomycin-resistant Enterococcus domination of intestinal microbiota is enabled by antibiotic treatment in mice and precedes bloodstream invasion in humans. J Clin Invest 120, 4332-4341 (2010).
42. Fukuda, S. et al. Bifidobacteria can protect from enteropathogenic infection through production of acetate. Nature 469, 543-547 (2011).
43. Yoshida, K. et al. Bifidobacterium response to lactulose ingestion in the gut relies on a solute-binding protein-dependent ABC transporter. Commun Biology 4, 541 (2021).
44. Bircher, J., Müller, J., Guggenheim, P. & Haemmerli, U. P. TREATMENT OF CHRONIC PORTAL-SYSTEMIC ENCEPHALOPATHY WITH LACTULOSE. Lancet 287, 890-893 (1966).
45. Elkington, S. G., Floch, M. H. & Conn, H. O. Lactulose in the Treatment of Chronic Portal-Systemic Encephalopathy-A Double-Blind Clinical Trial. New Engl J Medicine 281, 408-412 (1969).
46. Mao, B. et al. Lactulose Differently Modulates the Composition of Luminal and Mucosal Microbiota in C57BL/6J Mice. J Agr Food Chem 64, 6240-6247 (2016).
47. Karakan, T., Tuohy, K. M. & Solingen, G. J. Low-Dose Lactulose as a Prebiotic for Improved Gut Health and Enhanced Mineral Absorption. Frontiers Nutrition 8, 672925 (2021).
48. Bajaj, J. S. et al. Colonic mucosal microbiome differs from stool microbiome in cirrhosis and hepatic encephalopathy and is linked to cognition and inflammation. Am J Physiol-gastr L 303, G675-G685 (2012).
49. Riggio, O. et al. Effect of Lactitol and Lactulose Administration on the Fecal Flora in Cirrhotic Patients. J Clin Gastroenterol 12, 433-436 (1990).
50. Sim, K. et al. Improved Detection of Bifidobacteria with Optimised 16S rRNA-Gene Based Pyrosequencing. Plos One 7, e32543 (2012).
51. Yoshioka, H., Iseki, K. & Fujita, K. Development and differences of intestinal flora in the neonatal period in breast-fed and bottle-fed infants. Pediatrics 72, 317-21 (1983).
52. Henrick, B. M. et al. Bifidobacteria-mediated immune system imprinting early in life. Cell 184, 3884-3898.e11 (2021).
53. Vatanen, T. et al. Variation in Microbiome LPS Immunogenicity Contributes to Autoimmunity in Humans. Cell 165, 842-853 (2016).
54. Patole, S. K. et al. Benefits of Bifidobacterium breve M-16V Supplementation in Preterm Neonates-A Retrospective Cohort Study. Plos One 11, e0150775 (2016).
55. Sivan, A. et al. Commensal Bifidobacterium promotes antitumor immunity and facilitates anti-PD-L1 efficacy. Science 350, 1084-1089 (2015).
56. Matson, V. et al. The commensal microbiome is associated with anti-PD-1 efficacy in metastatic melanoma patients. Science 359, 104-108 (2018).
57. Xue, L. et al. Probiotics may delay the progression of nonalcoholic fatty liver disease by restoring the gut microbiota structure and improving intestinal endotoxemia. Sci Rep-uk 7, 45176 (2017).
58. Xu, R., Wan, Y., Fang, Q., Lu, W. & Cai, W. Supplementation with probiotics modifies gut flora and attenuates liver fat accumulation in rat nonalcoholic fatty liver disease model. J Clin Biochem Nutr 50, 72-77 (2011).
59. Sorbara, M. T. et al. Inhibiting antibiotic-resistant Enterobacteriaceae by microbiota-mediated intracellular acidification. J Exp Medicine 216, 84-98 (2019).
60. Yang, W. et al. Intestinal microbiota-derived short-chain fatty acids regulation of immune cell IL-22 production and gut immunity. Nat Commun 11, 4457 (2020).
61. Leclercq, S. et al. Intestinal permeability, gut-bacterial dysbiosis, and behavioral markers of alcohol-dependence severity. Proc National Acad Sci 111, E4485-E4493 (2014).
62. Bajaj, J. S., Kamath, P. S. & Reddy, K. R. The Evolving Challenge of Infections in Cirrhosis. New Engl J Med 384, 2317-2330 (2021).
63. Fernández, J. et al. Bacterial infections in cirrhosis: Epidemiological changes with invasive procedures and norfloxacin prophylaxis. Hepatology 35, 140-148 (2002).
64. Buffie, C. G. et al. Precision microbiome restoration of bile acid-mediated resistance to Clostridium difficile. Nature 517, 205-208 (2015).
65. Bajaj, J. S. et al. Association Between Intestinal Microbiota Collected at Hospital Admission and Outcomes of Patients With Cirrhosis. Clin Gastroenterol H 17, 756-765.e3 (2019).
66. Bajaj, J. S. et al. Association of serum metabolites and gut microbiota at hospital admission with nosocomial infection development in patients with cirrhosis. Liver Transplant 28, 1831-1840 (2022).
67. Biggins, S. W. et al. Diagnosis, Evaluation, and Management of Ascites, Spontaneous Bacterial Peritonitis and Hepatorenal Syndrome: 2021 Practice Guidance by the American Association for the Study of Liver Diseases. Hepatology 74, 1014-1048 (2021).
68. Wiest, R., Krag, A. & Gerbes, A. Spontaneous bacterial peritonitis: recent guidelines and beyond. Gut 61, 297 (2012).
69. Shamsaddini, A. et al. Impact of Antibiotic Resistance Genes in Gut Microbiome of Patients With Cirrhosis. Gastroenterology 161, 508-521.e7 (2021).
70. Piano, S. et al. Epidemiology and Effects of Bacterial Infections in Patients With Cirrhosis Worldwide. Gastroenterology 156, 1368-1380.e10 (2019).
71. Fernández, J., Bert, F. & Nicolas-Chanoine, M.-H. The challenges of multi-drug-resistance in hepatology. J Hepatol 65, 1043-1054 (2016).
72. Wong, F. et al. Clinical features and evolution of bacterial infection-related acute-on-chronic liver failure ⋆. J Hepatol 74, 330-339 (2021).
73. Dhiman, R. K. et al. Probiotic VSL #3 Reduces Liver Disease Severity and Hospitalization in Patients With Cirrhosis: A Randomized, Controlled Trial. Gastroenterology 147, 1327-1337.e3 (2014).
74. Holte, K., Krag, A. & Gluud, L. L. Systematic review and meta-analysis of randomized trials on probiotics for hepatic encephalopathy. Hepatol Res 42, 1008-1015 (2012).
75. Bajaj, J. S. et al. Long-term Outcomes of Fecal Microbiota Transplantation in Patients With Cirrhosis. Gastroenterology 156, 1921-1923.e3 (2019).
76. Bajaj, J. S. et al. Microbial Functional Change is Linked with Clinical Outcomes after Capsular Fecal Transplant in Cirrhosis. Jci Insight 4, (2019).
77. Bajaj, J. S. et al. Fecal microbiota transplant from a rational stool donor improves hepatic encephalopathy: A randomized clinical trial. Hepatology 66, 1727-1738 (2017).
78. DeFilipp, Z. et al. Drug-Resistant E. coli Bacteremia Transmitted by Fecal Microbiota Transplant. New Engl J Med 381, 2043-2050 (2019).
79. Zellmer, C. et al. Shiga Toxin-Producing Escherichia coli Transmission via Fecal Microbiota Transplant. Clin Infect Dis 72, e876-e880 (2020).
80. Bloom, P., Tapper, E. B., Young, V. B. & Lok, A. S. Microbiome Therapeutics for Hepatic Encephalopathy. J Hepatol 75, 1452-1464 (2021).
81. Liu, Q. et al. Synbiotic modulation of gut flora: Effect on minimal hepatic encephalopathy in patients with cirrhosis. Hepatology 39, 1441-1449 (2004).
82. Malaguarnera, M. et al. Bifidobacterium longum with Fructo-Oligosaccharide (FOS) Treatment in Minimal Hepatic Encephalopathy: A Randomized, Double-Blind, Placebo-Controlled Study. Digest Dis Sci 52, 3259 (2007).
83. Button, J. E. et al. Dosing a synbiotic of human milk oligosaccharides and B. infantis leads to reversible engraftment in healthy adult microbiomes without antibiotics. Cell Host Microbe 30, 712-725.e7 (2022).
84. Barratt, M. J. et al. Bifidobacterium infantis treatment promotes weight gain in Bangladeshi infants with severe acute malnutrition. Sci Transl Med 14, eabk1107 (2022).
85. Sinha, S. R. et al. Dysbiosis-Induced Secondary Bile Acid Deficiency Promotes Intestinal Inflammation. Cell Host Microbe 27, 659-670.e5 (2020).
86. Martino, C. et al. Acetate reprograms gut microbiota during alcohol consumption. Nat Commun 13, 4630 (2022).
87. Bolger, A. M., Lohse, M. & Usadel, B. Trimmomatic: a flexible trimmer for Illumina sequence data. Bioinformatics 30, 2114-2120 (2014).
88. Blanco-Miguez, A. et al. Extending and improving metagenomic taxonomic profiling with uncharacterized species with MetaPhlAn 4. Biorxiv 2022.08.22.504593 (2022) doi: 10.1101/2022.08.22.504593.
89. Li, D., Liu, C.-M., Luo, R., Sadakane, K. & Lam, T.-W. MEGAHIT: an ultra-fast single-node solution for large and complex metagenomics assembly via succinct de Bruijn graph. Bioinformatics 31, 1674-1676 (2015).
90. Buchfink, B., Reuter, K. & Drost, H.-G. Sensitive protein alignments at tree-of-life scale using DIAMOND. Nat Methods 18, 366-368 (2021).
91. Schluter, J. et al. The TaxUMAP atlas: Efficient display of large clinical microbiome data reveals ecological competition in protection against bacteremia. Cell Host Microbe (2023) doi: 10.1016/j.chom.2023.05.027.
92. Haak, B. W. et al. Impact of gut colonization with butyrate producing microbiota on respiratory viral infection following allo-HCT. Blood 131, blood-2018-01-828996 (2018).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating a drug-resistant pathogen in a patient, the method comprising administering a composition comprising lactulose and a commensal organism after the patient has been assayed for both a microbiome profile and a metabolic profile in a fecal sample from the patient.

2. (canceled)

3. The method of claim 1, wherein the drug-resistant pathogen comprises a vancomycin-resistant pathogen.

4. The method of claim 1, wherein the drug-resistant pathogen comprises an Enterococcus sp., an Enterococcus faecium, a Bifidobacteria sp. or a combination thereof.

5.-6. (canceled)

7. The method of claim 1, wherein the microbiome profile comprises a measured level of an Enterococcus sp., a Bifidobacteria sp., a Bacteroidetes sp., a Lachnospiraceae sp., a Proteobacteria sp., and/or a Lactobacillus sp.

8.-9. (canceled)

10. The method of claim 1, wherein the metabolic profile comprises a measured level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1.

11.-12. (canceled)

13. The method of claim 1, wherein the microbiome profile and the metabolic profile are determined based on a reference profile.

14.-19. (canceled)

20. The method of claim 1, wherein the lactulose is administered at a dose of between approximately 1-1,000 mg/L or 1-100 g/L.

21. The method of claim 1, wherein the commensal organism is administered at a dose of between approximately 1×10⁴ to 9×10⁹ colony forming units of the commensal organism.

22.-23. (canceled)

24. A method of treating a liver disease in a patient, the method comprising administering a composition comprising lactulose and a commensal organism after the patient has been assayed for both a microbiome profile and a metabolic profile in a fecal sample from the patient.

25. The method of claim 24, wherein the commensal organism comprises a Bifidobacteria sp.

26. The method of claim 24, wherein the microbiome profile comprises a measured level of an Enterococcus sp., a Bifidobacteria sp., a Bacteroidetes sp., a Lachnospiraceae sp., a Proteobacteria sp., and/or a Lactobacillus sp.

27.-28. (canceled)

29. The method of claim 24, wherein the metabolic profile comprises a measured level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1.

30.-31. (canceled)

32. The method of claim 24, wherein the microbiome profile and the metabolic profile are determined based on a reference profile.

33.-42. (canceled)

43. A composition comprising lactulose at a concentration of between approximately 1-1,000 mg/L or 1-100 g/L and a commensal organism of approximately 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×1013, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶ colony forming units.

44. The composition of claim 43, wherein the commensal organism comprises a Bifidobacteria sp.

45. The composition of claim 43, wherein the composition is formulated for oral administration.

46. The composition of claim 43, wherein the composition is formulated for administration to a human patient.

47. A method of measuring a microbiome profile and a metabolite profile in a sample, the method comprising measuring one or more of an Enterococcus sp., a Bifidobacteria sp., a Bacteroidetes sp., a Lachnospiraceae sp., a Proteobacteria sp., and/or a Lactobacillus sp. and measuring 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1.

48.-53. (canceled)

54. The method of claim 47, wherein the microbiome profile comprises a measured level of an Enterococcus sp., a Bifidobacteria sp., a Bacteroidetes sp., a Lachnospiraceae sp., a Proteobacteria sp., and/or a Lactobacillus sp.

55.-56. (canceled)

57. The method of claim 47, wherein the metabolic profile comprises a measured level of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, or 82 metabolites disclosed in Table 1.

58.-65. (canceled)
